SEQUENCE LISTING (58 sequences)

SEQ ID NO: 1 - DNA, 399 bp, Homo sapiens
ttagggttagggttagggttaggagaaaagggctgaacaactacctattggatactatggtccctacctgggtgatgggatcagtcataccccaaaccttagcatcatgcaatatacccatgtaacaaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaa

SEQ ID NO: 2 - DNA, 113 bp, Homo sapiens
ttagggttagggttaggggaatggaatcgaatcgaaaaaaaccctaaccctaaccctaaccctaaccctaaccctaaccctaaccctaaccctaaccctaaccctaaccctaa

SEQ ID NO: 3 - DNA, 361 bp, Homo sapiens
ttagggttagggttagggacagggtcttgctgtgttgcccaggctggagttgagtggcagtgatcacagctcactgcaccttgacctcccagactccccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaa

SEQ ID NO: 4 - DNA, 318 bp, Homo sapiens
ttagggttagggttagggttaggcgggaacccccgggcgcctgtggggtggtgtccgcgctcgcccccgcgtgggtgcggcgcgcgcctcccctattactcacctctcccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaa

SEQ ID NO: 5 - DNA, 233 bp, Homo sapiens
ttagggttagggttaggggaatggaatgcaatgcaaaaaaccctaaccctaaccctaaccctaaccctaaccctaaccctaaccctaaccctaaccctaaccctaaccctaaaatcctctccaactctgccactgcttaccaaataagtctatgaatattttaaatattttgttgttgttgtcatttcaacaatgttcacagtgtcttcacctaaccctaaccctaaccctaa

SEQ ID NO: 6 - DNA, 112 bp, Homo sapiens
ttagggttagggttaggggtcggggtcaggagaactaaatcactgaagagtcagacagtggaccccatcttgaaaaaaaaagaataacagaaagccctaaccctaaccctaa

SEQ ID NO: 7 - DNA, 277 bp, Homo sapiens
ttagggttagggttagggttccacctcccacatacagtcctgtatccttccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaa

SEQ ID NO: 8 - DNA, 311 bp, Homo sapiens
ttagggttagggttagggcgagggcgagggcgagggcgagggcgagggcgagggctaggttacttaactcagcctccatgtctttacctgcaaaatagggtacctctactcttgggctaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaa

SEQ ID NO: 9 - DNA, 135 bp, Homo sapiens
ttagggttagggttagggttagggttagggttagggttagggttagggttagggttagggcgggaacccccgggcgcctgtgggggaaggatgtgtgctcttatgaatgtgtgtaaaccctaaccctaaccctaa

SEQ ID NO: 10 - DNA, 343 bp, Homo sapiens
ttagggttagggttagggttatctctctctcttcccttctttcacaaatccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaa

SEQ ID NO: 11 - DNA, 300 bp, Homo sapiens
ttagggttagggttagggacagggtcttgctgtgttgcccaggctggagttgagtggctggagttgagtggcagtgatcacagctcactgcagccttgacctcccagactctgatcctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaa

SEQ ID NO: 12 - DNA, 197 bp, Homo sapiens
ttagggttagggttaggggaatggaatgcaatgcaaaaaaccctaaccctaaccctaaccctaaccctaaccctaaccctaaccctaaccctaaccctaagaatcccaagcccacacgttattaggctgcgcttcttaaaacaagttatgagatgggaaaaagggcacccacaaacataccctaaccctaaccctaa

SEQ ID NO: 13 - DNA, 252 bp, Homo sapiens
ttagggttagggttagggacctgagaagaactctgctccgcccttcgcaatacccccgaagtctgtgcagagccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaa

SEQ ID NO: 14 - DNA, 249 bp, Homo sapiens
ttagggttagggttagggttagggttagggttagggttagggttagggttagggttagggttagggttagggttagggttagggttagggttagggttagggttagggttagggttagggttagggttagggttagggttagggaaatatagaaagatagaggggaggttaaggtgcagtgagcgtgatcactgtcaccaattctagctgggcaacacagcaagaccctgtccctaaccctaaccctaa

SEQ ID NO: 15 - DNA, 144 bp, Homo sapiens
ttagggttagggttagggcgagggcgagggcgagggcgagggcgagggcgagggctaggttacttaactcagcctccatgtctttacctgcaaaatagggtgacgctgcctactgcttgggtctaaccctaaccctaaccctaa

SEQ ID NO: 16 - DNA, 305 bp, Homo sapiens
ttagggttagggttagggttagggttagggttagggttagggttagggttagggttagggttagggttagggttagggttagggttagggttagggttagggttagggttagggttagggttagggttagggttagggttagggttagggttagggttagggttagggttagggttagggttagggttagggttagggttagggttagggttagggttagggagagagagaggcacagcaggaggtctctcaactcgactgcgactcgccgtctcagccttccctaaccctaaccctaaccctaa

SEQ ID NO: 17 - DNA, 358 bp, Homo sapiens
ttagggttagggttagggaggctgaggcaggcggatcacgaggtcaggagattgagaccatcctggctaacatgatgaaacctcgtctctactaaaatacaaaaattacctaatgtggtggtggccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaaccctaa

SEQ ID NO: 18 - DNA, 149 bp, Homo sapiens
ttagggttagggttagggttagggttagggtaatcccatcactttgagaggtcaaggtgggaggactcgttgatcgacagtgacccagtcttgaaaaaagatacggaagacgactgccatgtgacctgtctccctaaccctaaccctaa

SEQ ID NO: 19 - DNA, 6 bp, Homo sapiens
ttaggg

SEQ ID NO: 20 - DNA, 18 bp, Homo sapiens
ttagggttagggttaggg

SEQ ID NO: 21 - DNA, 30 bp, Artificial Sequence
  PCR primer
gggnnngaattcttagggttagggttaggg

SEQ ID NO: 22 - DNA, 106 bp, Homo sapiens
agaaaagggctgaacaactacctattggatactatggtccctacctgggtgatgggatcagtcataccccaaaccttagcatcatgcaatatacccatgtaacaaa

SEQ ID NO: 23 - DNA, 23 bp, Homo sapiens
gaatggaatcgaatcgaaaaaaa

SEQ ID NO: 24 - DNA, 79 bp, Homo sapiens
acagggtcttgctgtgttgcccaggctggagttgagtggcagtgatcacagctcactgcaccttgacctcccagactcc

SEQ ID NO: 25 - DNA, 85 bp, Homo sapiens
cgggaacccccgggcgcctgtggggtggtgtccgcgctcgcccccgcgtgggtgcggcgcgcgcctcccctattactcacctctc

SEQ ID NO: 26 - DNA, 192 bp, Homo sapiens
gaatggaatgcaatgcaaaaaaccctaaccctaaccctaaccctaaccctaaccctaaccctaaccctaaccctaaccctaaccctaaccctaaaatcctctccaactctgccactgcttaccaaataagtctatgaatattttaaatattttgttgttgttgtcatttcaacaatgttcacagtgtcttca

SEQ ID NO: 27 - DNA, 76 bp, Homo sapiens
gtcggggtcaggagaactaaatcactgaagagtcagacagtggaccccatcttgaaaaaaaaagaataacagaaag

SEQ ID NO: 28 - DNA, 29 bp, Homo sapiens
ccacctcccacatacagtcctgtatcctt

SEQ ID NO: 29 - DNA, 101 bp, Homo sapiens
cgagggcgagggcgagggcgagggcgagggcgagggctaggttacttaactcagcctccatgtctttacctgcaaaatagggtacctctactcttgggcta

SEQ ID NO: 30 - DNA, 55 bp, Homo sapiens
cgggaacccccgggcgcctgtgggggaaggatgtgtgctcttatgaatgtgtgta

SEQ ID NO: 31 - DNA, 28 bp, Homo sapiens
tctctctctcttcccttctttcacaaat

SEQ ID NO: 32 - DNA, 97 bp, Homo sapiens
acagggtcttgctgtgttgcccaggctggagttgagtggctggagttgagtggcagtgatcacagctcactgcagccttgacctcccagactctgat

SEQ ID NO: 33 - DNA, 161 bp, Homo sapiens
gaatggaatgcaatgcaaaaaaccctaaccctaaccctaaccctaaccctaaccctaaccctaaccctaaccctaaccctaagaatcccaagcccacacgttattaggctgcgcttcttaaaacaagttatgagatgggaaaaagggcacccacaaacata

SEQ ID NO: 34 - DNA, 54 bp, Homo sapiens
acctgagaagaactctgctccgcccttcgcaatacccccgaagtctgtgcagag

SEQ ID NO: 35 - DNA, 87 bp, Homo sapiens
aaatatagaaagatagaggggaggttaaggtgcagtgagcgtgatcactgtcaccaattctagctgggcaacacagcaagaccctgt

SEQ ID NO: 36 - DNA, 108 bp, Homo sapiens
cgagggcgagggcgagggcgagggcgagggcgagggctaggttacttaactcagcctccatgtctttacctgcaaaatagggtgacgctgcctactgcttgggtctaa

SEQ ID NO: 37 - DNA, 59 bp, Homo sapiens
agagagagaggcacagcaggaggtctctcaactcgactgcgactcgccgtctcagcctt

SEQ ID NO: 38 - DNA, 106 bp, Homo sapiens
aggctgaggcaggcggatcacgaggtcaggagattgagaccatcctggctaacatgatgaaacctcgtctctactaaaatacaaaaattacctaatgtggtggtgg

SEQ ID NO: 39 - DNA, 101 bp, Homo sapiens
taatcccatcactttgagaggtcaaggtgggaggactcgttgatcgacagtgacccagtcttgaaaaaagatacggaagacgactgccatgtgacctgtct

SEQ ID NO: 40 - DNA, 24 bp, Artificial Sequence
  PCR Primer
gggnnngaattcttagggttaggg

SEQ ID NO: 41 - DNA, 18 bp, Artificial Sequence
  PCR primer
gggnnngaattcttaggg

SEQ ID NO: 42 - DNA, 12 bp, Homo sapiens
ttagggttaggg

SEQ ID NO: 43 - DNA, 24 bp, Homo sapiens
ttagggttagggttagggttaggg

SEQ ID NO: 44 - DNA, 6 bp, Artificial Sequence
  PCR primer
gggnnn

SEQ ID NO: 45 - DNA, 6 bp, Artificial Sequence
  PCR primer segment
nnnnnn

SEQ ID NO: 46 - DNA, 109 bp, Homo sapiens
atgattacaatttcaatttctaacctgttttattttgttttttttctgagacagggtctccctcttttgtccaaggctggagtgtggtagcgtatcacacgtgactcga

SEQ ID NO: 47 - DNA, 105 bp, Homo sapiens
tgcagtcagctgtgatagcactaccacactccagccttggacaacagagggagaccctgtctcagaaaaaaaaacaaaataaaacaggttagaaattgtatctgc

SEQ ID NO: 48 - DNA, 832 bp, Homo sapiens
tcagggtcagggtcagggttagggtcagggtaagggtcagggtacagcacttccgggttagggtcagggttagggtcagggtacagcacttccgggttagggtcagggttagggtcagggtcagggtacagcacttccgggttagggtcagggtcagggtcagggttagggtcagggtcagggtcagggtacagcacttccgggtcagggtcagggtcagggtcagggtcagggttagggtcagggtcagggtcagggtacagcatttccagcgctgccagcggcctccttgcttatggtgcatgttctaaccctcacaaaccacaagcttgtctttaaaaacatcaagttgaaataaaccacatattaattgaggtaaaataagtggccagagaacccacataatttagttgcagtaaacttctgctgcatatttaaaggaaaataaacgaaataatagttttcaaaacataaaaattattccactctttctgaaaacacactgctaatctaagcctaaccaaaaagctaaccctaaccctaccactaaccctaatgctaccactagcctctaaccctactgctaactctaacacctaaccctaatcccaaacccctaaaccaaaccctagtcctgaaccctaaccctaaccccaacccaaaccccaataccgacactacactaatcataacccaaccctaactctaaccgcaaaaccctaacccctgaccctaaacccaaccccaacccaaaacctaactctaacccctgaaccaaaccctaaaaccccacaaaccccaaaaatattccaaaccgaacctcaccctaacc

SEQ ID NO: 49 - DNA, 592 bp, Homo sapiens
tttgggttagagttagagttagagttagagttagagttagagttagagttaggaatgtcaaccccattgtcagtggctgactctcagttgcccatccctggttcagggttaaagttagggtttgggttagagttagagttagagttagagttagagttagagttaggaatgtcaaccccattgtcagtggctgactctcagttgcccatccctggttcagggttagggttagggttaggtttaggaaagtcagccccattgcagttcctgactcacagttgctcatccatgggaaactcctactgtcaccagagatggtccaagcagggccctggtgaagttccccaggcctgcattctctgtaactcggatgagctcagaagggcttgaaatctctggtcaaaatcacaatgaggaatgaggaggacaaagcccttgcctgggcccctccttcatccaggaggactggcgcaaagaacagtggctcccggagagcttgggagctgatttttaacagtcaatgtctttccaggtcaaccacctttttaaatttttttcaggaatagaataaaaaacggtcttgaccaacttc

SEQ ID NO: 50 - DNA, 174 bp, Homo sapiens
tcagggttatggtcagggtcagggtcagggtcagggtcagggtcagggtcagggtcagggttagggttagggtcagggtcagggtcagggtcagggtcagggtcagggtcagggtcagggtcagggtcagggtcagggttagggtcagggtcagggtcagggtcagggtcaggg

SEQ ID NO: 51 - DNA, 106 bp, Homo sapiens
tttgttacatgggtatattgcatgatgctaaggtttggggtatgactgatcccatcacccaggtagggaccatagtatccaataggtagttgttcagcccttttct

SEQ ID NO: 52 - DNA, 67 bp, Homo sapiens
caccccaagcagtaggcagcgtcacccctattttgcaggtaaagacatggaggctgagttaagtaac

SEQ ID NO: 53 - DNA, 36 bp, Homo sapiens
tcgccctcgccctcgccctcgccctcgccctcgccc

SEQ ID NO: 54 - DNA, 230 bp, Homo sapiens
ctagggctagggctagggctagggctagggctagggttagggtcagggtcagggtcagggtcagggtcagggtcagggtcagggtcagggttttagggttagggttagggttagggttagggtcagggtcagggtcagggttagggtcagggtcagggtcagggtcagggttaagggtcagggtcagggtcagggtcagggtcagggtcaggggtaggggtaggggtagg

SEQ ID NO: 55 - DNA, 114 bp, Homo sapiens
gcgcccaccaccacacttggctaattttttgtatttttagtagagacgaggtttcatcatgttagccaggatggtcccaatctcctgacctcgtgatccgcctgcctcagcctc

SEQ ID NO: 56 - DNA, 280 bp, Homo sapiens
tcagggttatggtcagggtcagggtcagggtcagggtcagggtcagggtcagggtcagggttagggttagggtcagggtcagggtcagggtcagggtcagggtcagggtcagggtcagggtcagggtcagggtcagggttagggtcagggtcagggtcagggtcagggtcagggtttgttacatgggtatattgcatgatgctaaggtttggggtatgactgatcccatcacccaggtagggaccatagtatccaataggtagttgttcagcccttttct

SEQ ID NO: 57 - DNA, 103 bp, Homo sapiens
caccccaagcagtaggcagcgtcacccctattttgcaggtaaagacatggaggctgagttaagtaactcgccctcgccctcgccctcgccctcgccctcgccc

SEQ ID NO: 58 - DNA, 344 bp, Homo sapiens
ctagggctagggctagggctagggctagggctagggttagggtcagggtcagggtcagggtcagggtcagggtcagggtcagggtcagggttttagggttagggttagggttagggttagggtcagggtcagggtcagggttagggtcagggtcagggtcagggtcagggttaagggtcagggtcagggtcagggtcagggtcagggtcaggggtaggggtaggggtagggcgcccaccaccacacttggctaattttttgtatttttagtagagacgaggtttcatcatgttagccaggatggtcccaatctcctgacctcgtgatccgcctgcctcagcctc

(19) United States
(12) Patent Application Publication
(10) Pub. No.: US 2009/0029359 A1
(43) Pub. Date: Jan. 29, 2009

(54) DIAGNOSTIC METHODS FOR EARLY CANCER DETECTION

(75) Inventors: David P. Gilley, Carmel, IN (US); Nazmul Huda, Indianapolis, IN (US)

Correspondence Address: BARNES & THORNBURG LLP, 11 SOUTH MERIDIAN, INDIANAPOLIS, IN, 46204, US

(73) Assignee: Indiana University Research and Technology Corporation, Indianapolis, IN (US)

(21) Appl. No.: 11/816,363

(22) PCT Filed: Feb. 15, 2006
(86) PCT No.: PCT/US06/05284

§ 371 Date: Sep. 11, 2008

Related U.S. Application Data
(60) Provisional application No. 60/654,320, filed on Feb. 18, 2005.

(51) Int. Cl. C12Q 1/68; C07H 21/04

(52) U.S. Cl. 435/6; 536/23.1; 536/24.33; 536/24.31

(57) ABSTRACT

The present disclosure is directed to compositions and methods for detecting signs of telomere dysfunction as diagnostic indicators of metastatic disease. More particularly, diagnostic reagents and procedures are provided for analyzing samples to detect elevated expression of TRK2 protein or detect the presence of telomere fusions as an early diagnostic test for cancerous or pre-cancerous cells. In one embodiment the methods of the present disclosure are used to diagnose the existence of, or assess the risk of, breast cancer in an individual.

### RELATED APPLICATIONS

This application claims priority under 35 USC § 119(e) to U.S. Provisional Application Ser. No. 60/654,320, filed Feb. 18, 2005, the disclosure of which is incorporated herein by reference.

### BACKGROUND

Genomic instability is one of the earliest neoplastic changes known to occur in tumorigenesis. Several recent reports indicate that defects in telomere maintenance may play an important role in the development of cancer, and more particularly, breast cancer. Telomeres are specialized DNA/protein structures, functioning as protective caps to prevent chromosome end fusions and permit DNA end replication. As cells divide, the length of the telomeres shrinks until the telomere reaches a critical size wherein the cell stops dividing.

Telomerase is a ribonucleoprotein complex consisting of a reverse transcriptase catalytic subunit (hTERT) and an RNA (hTER) that supplies the template for (T₂AG₃) repeat addition, among other essential functions. Telomerase is absent, or greatly reduced, in most somatic cells resulting in progressive telomere shortening after each cell cycle that can lead to loss of telomere function. Telomerase is activated early in the progression of breast cancer, and is present in ductal carcinoma in situ (DCIS). Remarkably, telomerase is activated in over 95% of tumors via a complex process, including loss of hTERT-specific negative transcription regulators, likely caused by earlier events of genomic instability and hTERT-specific positive transcription activators. Therefore, hTERT activation is the most frequent gene regulatory alteration known to occur in most cancers and potentially an extremely useful marker. However, there are likely earlier events that are also associated with tumorigenesis that may provide for earlier detection of cancerous or pre-cancerous cells.

A critical component of mammalian telomere maintenance involves the correct tissue-specific regulation of telomere DNA length. However, just as importantly, the proper regulation of the proteinaceous telomere cap must be maintained with its own set of unique tissue and developmental complexities. Telomere-associated proteins, such as TRF1 and TRF2, can bind telomeric DNA directly or can localize to the telomere via interactions with telomere repeat binding proteins. Interactions between telomere-associated proteins and telomeric DNA, as well as telomere repeat synthesis by telomerase are critical for the maintenance of telomere length and capping function throughout development and the cell cycle. Adding an additional layer of complexity, human telomeres end in a 3′ G-rich single-strand overhang consisting of several hundred nucleotides that can displace one strand of the telomeric repeat and hybridize to its complementary sequence. The resulting structure of a large duplex loop, called the t-loop, contains the folded DNA and associated proteins, particularly TRF2, which is thought to bind to the t-loop junction.

Several studies have reported that the artificial overexpression of wild type and dominant negative alleles of TRF1 and TRF2 results in progressive telomeric DNA shortening and elongation, respectively (see for example Smogorzewska and de Lange, (2004) Curr Biol, 12, 1635-44). Complete deficiency of telomere-associated proteins generally results in shortening of telomeric DNA length and loss of capping function resulting in the accumulation of telomere fusions (Ferreira et al., (2004) Mol Cell. January 16;13(1):7-18.; Smogorzewska and de Lange, 2004).

Telomere dysfunction, caused by critical short telomeric DNA or other telomere maintenance defects, may be an early event causing genomic instability during the progression of breast cancers (Artandi and DePinho, (2000) Nat Med. August;6(8):852-5). Telomere dysfunction induced in mice by disruption or up-regulation of telomerase activity results in high levels of breast adenocarcinomas and other epithelial cancers not normally found in these strains of mice. Additionally, normal human mammary epithelial cells (HMECs) can spontaneously escape senescence and acquire genomic alterations including telomere fusions. The prevention of chromosome end-to-end fusions by functional telomere caps and the regulation of telomere DNA replication are critical components in the maintenance of genomic integrity. Loss of telomere capping allows chromosome ends to fuse, causing breakage-fusion-bridge cycles, resulting in genomic instability.

Telomere length can be readily determined in tissue, however, telomere shortening does not necessarily indicate loss of telomere function, and telomere dysfunction can occur without telomeric DNA shortening. Hence, the methodology of the present disclosure allows for the examination of the loss of telomere function during breast tumorigenesis and the detection of such loss as an early diagnostic of cancerous or pre-cancerous cells. The extent of telomere dysfunction in human breast tumorigenesis has not been reported.

### SUMMARY

The present disclosure is directed to diagnostic reagents and procedures for the early detection of cancer. More particularly, the present disclosure is directed to methods for analyzing samples to assess the existence of cancerous or pre-cancerous cells. In one embodiment the methods of the present disclosure are used to diagnose the existence of, or assess the risk of, breast cancer.

In accordance with one embodiment, a method of detecting telomere fusions in a biological sample as a diagnostic indicator of the existence of cancerous or pre-cancerous cells is provided. The method comprises contacting cellular DNA isolated from a biological sample with a telomere specific PCR primer to form a reaction substrate and conducting a PCR amplification reaction on the reaction substrate. The biological sample may be purified DNA from a patients cells or the biological sample may be thin slices of cells or a tissue sample obtained from a patient. After conducting the PCR amplification the sample is screened for the presence of amplified products, wherein the detection of an amplified product indicates the presence of telomere fusions and thus is diagnostic for the presence of cancerous or pre-cancerous cells in the patient's tissue.

In another embodiment a kit is provided for screening biological samples for the presence telomere fusions. More particularly the kit comprises a telomere specific PCR primer. In one embodiment the isolated PCR primer comprises the sequence of SEQ ID NO: 19. The kit can be further provided with one or more reagents for conducting PCR reactions or for detecting the amplified products produced by the PCR reaction. In one embodiment the PCR primer comprises a sequence represented by the general formula X-Y-(Z)n, wherein X represents a sequence of six nucleotides, Y represents a restriction endonuclease recognition sequence, Z represents the sequence of SEQ ID NO: 19, and n is an integer selected from the range of 1-6.

In another embodiment, a method of detecting aberrant TRK2 expression in a patient's cells, as a diagnostic indicator of the presence of cancer or pre-cancerous cells, is provided The method comprises contacting proteins of the patient's tissue with an ligand that specifically binds to TRK2, detecting specific ligand-TRK2 complexes, and comparing the expression of TRK2 protein in the patient's tissue to that of normal cells to detect aberrant TRK2 expression in the tissue sample. In one embodiment the ligand is a monoclonal antibody specific for TRK2 and the aberrant expression may constitute a significant elevation in the amount of TRK2 protein present, and/or the cyto-location of the TRK2 protein.

In a further embodiment, a method of detecting telomere fusions associated with neoplastic cells is provided that utilizes telomere specific nucleic acid probes. In one embodiment the probe comprises a composition that includes one, or a combination of two or more, of the sequence SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57 and SEQ ID NO: 58.

### BRIEF DESCRIPTION OF THE DRAWINGS

FIGS. 1A & 1B provide data showing that the TRF2 protein is significantly increased in immortally transformed human mammary epithelial cells (HMECs). FIG. 1A is a schematic drawing showing the generation of immortal HMEC lines. Primary cultures of 184 HMEC exposed to the chemical carcinogen benzo(a)pyrene [B(a)P] gave rise to extended life span cultures lacking p16 expression. Rare immortally transformed lines emerged from extended life span 184Aa or 184Be either spontaneously, following insertional mutagenesis, inactivation of p53 function, and/or transduction of breast cancer-associated oncogene ZNF217. Rare immortally transformed lines emerged from unexposed post-selection p16(−) 184 HMEC following transduction of breast cancer associated oncogene c-myc. See text and web site (www.lbl.gov/˜mrgs/mindex.html) for more details. FIG. 1B represents the quantitation of immunoblot data showing up-regulation of TRF2 protein in independently derived immortal HMEC lines. Pixel densities for TRF2 and TIN2 bands were divided by those for the control bands, and plotted relative to the levels in 184Aa.

FIG. 2. is a bar graph representing data generated from immunoblots of protein samples isolated from breast tumor derived cell lines that were probed using an anti-TRF2 antibody. Signal intensities for TRF2 bands were divided by those for the control bands, and plotted relative to the levels in the 184 cells.

FIGS. 3A & 3B demonstrate the effect of exogenously introduced TRF2 genes on cumulative population doublings achieved prior to agonescence/crisis. The transduced cells were grown in the presence of selective drugs to confluence, then replated in triplicate at a fixed density of 1×10⁵/60 mm dish. The total number of cells harvested at every subculture was calculated and the number of accumulated population doublings (PD) per passage was determined using the equation, PD=(A/B)/log2, where A is the number of harvested cells, and B is the number of plated cells, not corrected for plating efficiency. Experiments were terminated when the cultures failed to achieve confluence within 3 weeks. Each experiment was repeated three times and in each case representative data from one experiment is shown.

FIG. 4 is a schematic representation of the events that occur resulting in a telomere fusion between two chromosomes, leading to genomic instability. The first step is an uncapping event followed by fusion between two uncapped chromosomes.

FIG. 5 is a schematic representation of a fusion junction, demonstrating the binding of telomere specific primers that enable PCR amplification of telomere fusion junction. Nₙ represents a variable number of nucleotides added to the 5′ end of the PCR primer to optimize the specificity of the amplification reaction, wherein n is an integer selected from the range of 1 to 6.

FIG. 6 represents a Southern blot of PCR products produced using a telomere specific primer and a template comprising a positive control, negative control, and DNA isolated from BJ cells and BJ HVP E6/E7 cells, respectively. The blots were probed using a ³²P labeled (TTAGGG)₄ (SEQ ID NO: 43) oligonucleotide probe at the designated three annealing temperatures.

FIG. 7 represents the results of a sequence analysis of the cloned telomere fusion junctions from BJ HVP E6/E7 cells. The clones contained various sizes of telomeric repeats, and fragments of non-telomeric DNA (23-194 bp) are inserted between telomere-telomere fusions in all clones examined to date. The non-telomeric DNA found inserted at the telomere fusion sites, in 37 out of 40 clones sequenced, corresponds to previously identified fragile chromosome sites as indicated. The fragile chromosome sites indicated in bold represent telomere fusions have also been cloned and sequenced from a breast tumor tissue sample.

FIG. 8 represents a model for the generation of telomere fusion junctions having an internal fragile site fragment.

### DETAILED DESCRIPTION

### Definitions

In describing and claiming the invention, the following terminology will be used in accordance with the definitions set forth below.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

The term “isolated” as used herein refers to material that has been removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

As used herein, the term “purified” and like terms relate to the isolation of a molecule or compound in a form that is substantially free of contaminants normally associated with the molecule or compound in a native or natural environment.

As used herein, the term “primer” refers to an oligonucleotide, whether occurring naturally as in a purified restriction digest product, or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products.

The term “thermostable polymerase” refers to a polymerase enzyme that is heat stable, i.e., the enzyme catalyzes the formation of primer extension products complementary to a template and does not irreversibly denature when subjected to the elevated temperatures for the time necessary to effect denaturation of double-stranded template nucleic acids. Generally, the synthesis is initiated at the 3′ end of each primer and proceeds in the 5′ to 3′ direction along the template strand. Thermostable polymerases have been isolated from Thermus flavus, T. ruber, T. thermophilus, T. aquaticus, T. lacteus, T. rubens, Bacillus stearothermophilus, and Methanothermus fervidus. Nonetheless, polymerases that are not thermostable also can be employed in PCR assays provided the enzyme is replenished.

As used herein, the term “antibody” refers to a polyclonal or monoclonal antibody or a binding fragment thereof such as Fab, F(ab′)2 and Fv fragments.

The term “label” as used herein refers to any atom or molecule which can be used to provide a detectable (preferably quantifiable) “signal”, and which can be attached to a nucleic acid or protein. Labels may provide “signals” detectable by fluorescence, radioactivity, colorimetry, gravimetry, X-ray diffraction or absorption, magnetism, enzymatic activity, and the like.

The term “restriction endonuclease” as used herein refers to enzymes that cleave the phosphodiester bond of a deoxyribonucleic acid (DNA) chain at specific sites within the restriction endonuclease recognition sequence (i.e. the “Type II restriction endonucleases).

The term “restriction endonuclease recognition sequence” refers to a nucleic acid sequence that is the target site for cleavage by a restriction endonuclease.

The term “neoplastic cells” as used herein refers to cells that result from abnormal new growth.

As used herein, the term “tumor” refers to an abnormal mass or population of cells that result from excessive cell division, whether malignant or benign, and all pre-cancerous and cancerous cells and tissues. A “tumor” is further defined as two or more neoplastic cells.

“Malignant cells/tumors” are distinguished from benign cells/tumors in that, in addition to uncontrolled cellular proliferation, they will invade surrounding tissues and may additionally metastasize. Cancer cells are cells that have undergone malignant transformation

The term “neoplastic disease” as used herein refers to a condition characterized by uncontrolled, abnormal growth of cells. Neoplastic diseases include cancer.

### EMBODIMENTS

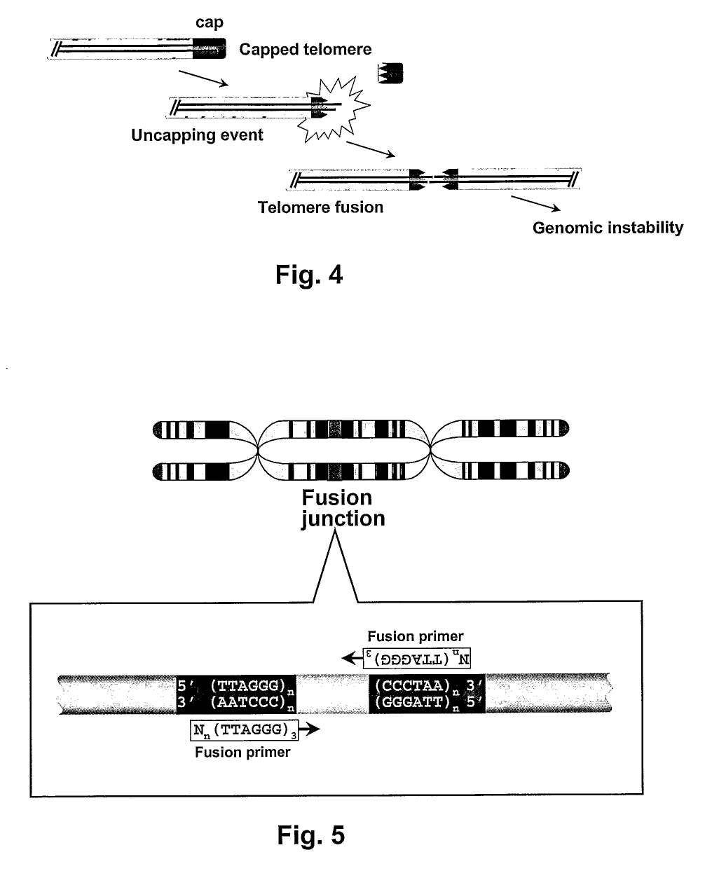

The present disclosure is based on the premise that telomere dysfunction is a driving force behind the genomic instability observed in early malignant lesions. The telomere dysfunction hypothesis states that telomere capping is disrupted in a small subset of normal precursor cells, leading to telomere fusions that ultimately cause genomic instability via breakage-fusion-bridge cycles (FIG. 4). Consequently, loss of genomic integrity leads to mis-regulation of genes (including TERT—the catalytic component of telomerase) involved in growth control, ultimately resulting in tumorigenesis.

One aspect of the present disclosure is directed to compositions and methods for detecting signs of telomere dysfunction as diagnostic indicators of metastatic disease. More particularly, in accordance with one embodiment a method is provided for detecting the presence of telomere fusion products in the cells of a biological sample. Such telomere fusions have been associated with cancerous or pre-cancerous cells and can serve as early diagnostic markers of neoplastic disease.

In accordance with one embodiment, the method of detecting telomere fusions in the cells of a biological sample comprises the use of a standard PCR reaction to specifically amplify any telomeric fusion product present in a biological sample. In one embodiment the biological sample represents nucleic acid sequences isolated from a patient's tissues, and more particularly in one embodiment the patient is a human. The tissue may comprise a blood sample or a solid tissue biopsy sample recovered from the patient. In one embodiment the biological sample represent human breast tissue. Specific amplification of telomere fusions is accomplished through the use of a telomere specific PCR primer and appropriate reaction conditions. Absent the presence of a telomere fusion product, the PCR reaction will fail to produce an amplicon through the use of the telomere specific PCR primer disclosed herein.

In accordance with one embodiment a nucleic acid sequence is provided that can serve as a telomere specific primer for amplifying telomere fusion products. In one embodiment the telomere specific PCR primer comprises the sequence of SEQ ID NO: 19. In one embodiment the primer comprises tandem repeats of the sequence of SEQ ID NO: 19 ranging anywhere from about 1 to about 6 repeats. In one embodiment the primer comprises 2 to 3 tandem repeats of SEQ ID NO: 19 and in one embodiment the primer comprises the sequence of SEQ ID NO: 20.

The telomere specific PCR primer can be further provided with a restriction endonuclease recognition sequence to assist in the cloning of the amplified telomere fusion region. In accordance with one embodiment a purified nucleic acid sequence is provided, comprising SEQ ID NO: 19 and a restriction endonuclease recognition sequence, wherein the restriction endonuclease recognition sequence is covalently linked to the 5′ end of SEQ ID NO: 19. In one embodiment the telomere specific PCR primer comprises a sequence represented by the general formula X-Y-(Z)ₙ, wherein X represents the sequence NNNNNN (SEQ ID NO:45), and in one embodiment X represents the sequence GGGNNN (SEQ ID NO: 44), wherein N represents any of the four standard nucleotides (guanosine, cytidine, thymidine or adenosine), Y represents a restriction endonuclease recognition sequence, Z represents the sequence of SEQ ID NO: 19, and

n is an integer selected from the range of 1-6. In one embodiment n is an integer selected from the range of 2 to 4 or 2 to 3. In one embodiment n is 3. In one embodiment the restriction endonuclease recognition sequence comprises a recognition sequence of six nucleotides. In one embodiment the restriction endonuclease recognition sequence is a recognition sequence for an enzyme selected from the group consisting of EcoRI, BamHI, HindIII, PstI, KpnI, PvuII, ApaI, HpaI, SalI, ClaI, XbaI, BglII. In one embodiment, the restriction endonuclease recognition sequence is the recognition site for EcoRI. In one embodiment the telomere specific PCR primer comprises the sequence GGGNNNGAATTC(TTAGGG)ₙ (SEQ ID NO: 21, SEQ ID NO: 40 and SEQ ID NO: 41), wherein n is an integer selected from the range of 1-3, and which includes the recognition sequence for the endonuclease EcoRI. In a further embodiment the PCR primer consists of SEQ ID NO: 21.

In accordance with one embodiment, a method of detecting the presence of telomere fusions in a population of cells is provided using a PCR reaction and the telomere specific primer disclosed herein. The method comprises the steps of contacting a biological sample with a telomere specific primer, conducting a PCR amplification reaction, and screening the sample to detect the presence of an amplified product. In accordance with one embodiment the biological sample comprises total DNA, or nuclear DNA, recovered from mammalian cells. In another embodiment the biological sample comprises thin sections of mammalian cells/tissues.

The cellular DNA of the biological sample is contacted with the telomere specific PCR primer under conditions that allow the PCR primer to bind to its complementary strand on the target DNA, to form a reaction substrate. Suitable buffers and polymerase enzymes are then added to the reaction substrate, and a PCR amplification reaction is run using standard techniques known to those skilled in the art. Advantageously, due to the symmetry of the telomere fusions only a single PCR primer is required for amplification of the telomere fusion (see FIG. 5). In the absence of a telomere fusion, no DNA amplification will occur. In one embodiment the PCR primer used comprises the sequence of SEQ ID NO: 19, and more particularly, in one embodiment the primer comprises a sequence represented by the general formula X-Y-(Z)ₙ, wherein X represents a nucleic acid sequence of six nucleotides, Y represents a restriction endonuclease recognition sequence, Z represents the sequence of SEQ ID NO: 19, and n is an integer selected from the range of 1-6. In one embodiment n is an integer selected from the range of 1 to 3 or 2 to 3, and X represents the sequence GGGNNN (SEQ ID NO: 44), wherein N represents any of the four standard nucleotides (guanosine, cytidine, thymidine or adenosine).

After completion of the PCR amplification reaction the sample is screened for the presence of an amplified product. Detection of the amplification product can be conducted using any of the known techniques used to detect the presence of nucleic acid sequences. In accordance with on embodiment the PCR primer is labeled with a detectable marker, and the production of a PCR amplicon can be detected based on the detection of a labeled nucleic acid amplified product that is larger in size than the original PCR primer. Alternatively, in one embodiment the PCR primer is not labeled and the amplification products are detected through the use of DNA intercalating agents (such as ethidium bromide), or other DNA binding entities that are labeled or produce a signal upon binding to DNA. An additional example includes the use of standard Southern blotting techniques known to those skilled in the art to detect the amplified product. Detection of an amplified product (i.e. a DNA segment greater in size then the original primer) indicates the presence of telomere fusions in the original cells of the biological sample. In accordance with one embodiment the amplified sequences can be cloned and sequenced to provide further information regarding the detected telomere fusions.

The tissue selected for analysis can be any mammalian tissue and in one embodiment any human tissue. In one embodiment the tissue represents a biopsy sample recovered from a human and in one embodiment the sample is human female breast tissue. In one embodiment nuclei acid sequences are first isolated from the cells of the biological sample. More particularly, DNA, including total DNA or in one embodiment, genomic DNA is isolated from the cells. This isolated DNA is then contacted with the telomere specific PCR primer and the PCR amplification reaction is conducted. In an alternative embodiment the PCR reaction can be conducted in situ on thin slices of the biological tissue sample. In situ reactions offer the advantage of demonstrating the extent of telomere fusions in the cells of the tissue sample and may provide prognostic information as well as help define treatment strategies.

As described in Example 3, the present disclosure has demonstrated a PCR based methodology can be used to specifically amplify telomere-telomere fusions present in DNA isolated from cells that contain known percentages of telomere fusions. These telomere fusion PCR products were cloned and sequenced to determine the DNA sequence at the fusion junction sequences. Table 1 represents fusion junction sequences isolated from BJ foreskin fibroblasts (BJ HPV E6/E7 cells), whereas Tables 2 and 3 represent fusion junction sequences isolated from human breast tumor tissue. Table 2 represents fusion sequences from Class I breast tumor tissue fusion junctions, wherein the fusion junction sequence is associated with a single known fragile site. Table 3 represents fusion sequences from Class II breast tumor tissue fusion junctions, wherein the fusion junction sequence represents two regions of sequences (A and B) wherein each fusion junction sequence region is associated with a separate and distinct known fragile site.

The telomere fusions disclosed in Tables 1, 2 and 3 represent unique combination of native nucleic acid sequences. Accordingly, nucleic acid probes specific for these sequences could potential serve as markers for detecting the presence of telomere fusions in cells. In accordance with one embodiment a composition for detecting telomere fusion products is provided wherein the composition comprises a labeled nucleic acid sequence selected from the group consisting of SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57 and SEQ ID NO: 58. In accordance with one embodiment a composition comprising two or more of the nucleic acid sequence selected from the group consisting of SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57 and SEQ ID NO: 58 is used as probes for detecting the presence of such sequences (or their complementary sequence) in a population of cells. Detection of one or more of the sequences of SEQ ID NOs 22-39 in a biological sample recovered from a patient is anticipated to be diagnostic for the presence of cancerous or pre-cancerous cells.

TABLE 1
Sequencing Data

| | | | Size of | | |
|---|---|---|---|---|---|
| | No. of Telomeric | Non-Telomeric Sequence | non-telomeric | | No. of Telomeric |
| Sample | Repeats (Right) | at Fusion Junctions | Sequence (bp) | Origin | Repeats (Left) |
| 1. | (TTAGGG)₃TTAGG | AGAAAAGGGCTGAACAACTACCTATTGGATA | 106 | Chr. 2 | (CCCTAA)₄₅ |
| (SEQ ID | | CTATGGTCCCTACCTGGGTGATGGGATCAGT | | | |
| NO: 1) | | CATACCCCAAACCTTAGCATCATGCAATATA | | | |
| | | CCCATGTAACAAA | | | |
| 2. | (TTAGGG)₃ | GAATGGAATCGAATCGAAAAAAA | 23 | Chr. 10/17 | (CCCTAA)₁₂ |
| (SEQ ID | | | | | |
| NO:2) | | | | | |
| 3. | (TTAGGG)₃ | ACAGGGTCTTGCTGTGTTGCCCAGGGTGGAG | 79 | Chr. 8 | (CCCTAA)₄₄ |
| (SEQ ID | | TTGAGTGGCAGTGATCACAGCTCACTGCACC | | | |
| NO:3) | | TTGACCTCCCAGACTCC | | | |
| 4. | (TTAGGG)₃TTAGG | CGGGAACCCCCGGGCGCCTGTGGGGTGGTGT | 85 | Human | (CCCTAA)₃₅ |
| (SEQ ID | | CCGCGCTCGCCCCCGCGTGGGTGCGGCGCGC | | ribosomal | |
| NO:4) | | GCCTCCCCTATTACTCACCTCTC | | DNA | |
| 5. | (TTAGGG)₃ | GAATGGAATGCAATGCAAAAAA(CCCTAA)₁₂ | 22-(CCCTAA)₁₂-95 | Chr. 7 + | CCTAA(CCCTAA)₃ |
| (SEQ ID | | AATCCTCTCCAACTCTGCCACTGCTTACCAAA | | Chr. 6 | |
| NO:5) | | TAAGTCTATGAATATTTTAAATATTTTGTTGT | | | |
| | | TGTTGTCATTTCAACAATGTTCACAGTGTCTT | | | |
| | | CA | | | |

| | |
|---|---|
| 6. | Both of the samples, 5 and 6, are harboring the inserts of same sequence |

| | | | | | |
|---|---|---|---|---|---|
| 7. | (TTAGGG)₃ | GTCGGGGTCAGGAGAACTAAATCACTGAAGA | 76 | Chr. 11 | (CCCTAA)₃ |
| (SEQ ID | | GTCAGACAGTGGACCCCATCTTGAAAAAAAA | | | |
| NO:6) | | AGAATAACAGAAAG | | | |
| 8. | (TTAGGG)₃TT | CCACCTCCCACATACAGTCCTGTATCCTT | 29 | Chr. 2 | (CCCTAA)₃₈ |
| (SEQ ID | | | | | |
| NO:7) | | | | | |
| 9. | (TTAGGG)₃ | (CGAGGG)6CTAGGTTACTTAACTCAGCCTC | 101 | Chr. 3 + | (CCCTAA)₃₂ |
| (SEQ ID | | CATGTCTTTACCTGCAAAATAGGGTACCTCT | | Chr. 4 | |
| NO:8) | | ACTCTTGGGCTA | | | |
| 10. | (TTAGGG)₁₀ | CGGGAACCCCCGGGCGCCTGTGGGGGAAGGA | 57 | Chr. 11 | AA(CCCTAA)₃ |
| (SEQ ID | | TGTGTGCTCTTATGAATGTGTGTA | | | |
| NO:9 | | | | | |
| 11. | (TTAGGG)₃TTA | TCTGTCTGTCTTCCCTTCTTTCACAAAT | 28 | Chr. 7 | (CCCTAA)₄₉ |
| (SEQ ID | | | | | |
| NO:10) | | | | | |
| 12. | (TTAGGG)₃ | ACAGGGTCTTGCTGTGTTGCCCAGGCTGGAG | 97 | Chr. 8 | CCTAA(CCCTAA)₃₀ |
| (SEQ ID | | TTGAGTGGCTGGAGTTGAGTGGCAGTGATCA | | | |
| NO:11) | | CAGCTCACTGCAGCCTTGACCTCCCAGACTC | | | |
| | | TGAT | | | |
| 13. | (TTAGGG)₃ | GAATGGAATGCAATGCAAAAAA(CCCTAA)₁₀ | 22-(CCCTAA)₁₀-80 | Chr. 7 + | (CCCTAA)₃ |
| (SEQ ID | | GAATCCCAAGCCCACACGTTATTAGGCTGCGC | | Chr. 22 | |
| NO:12) | | TTCTTAAAACAAGTTATGAGATGGGAAAAAGG | | | |
| | | GCACCCACAAACATA | | | |
| 14. | (TTAGGG)₃ | ACCTGAGAAGAACTCTGCTCCGCCCTTCGCAA | 54 | Chr. 10 | (CCCTAA)₃₀ |
| (SEQ ID | | TACCCCCGAAGTCTGTGCAGAG | | | |
| NO:13) | | | | | |
| 15. | (TTAGGG)₂₄ | AAATATAGAAAGATAGAGGGGAGGTTAAGGT | 89 | Chr. 8 | (CCCTAA)₃ |
| (SEQ ID | | GCAGTGAGCGTGATCACTGTCACCAATTCTA | | | |
| NO:14) | | GCTGGGCAACACAGCAAGACCCTGT | | | |
| 16. | (TTAGGG)₃ | (CGAGGG)6CTAGGTTACTTAACTCAGCCTC | | Chr. 3 + | (CCCTAA)₃ |
| (SEQ ID | | CATGTCTTTACCTGCAAAATAGGGTGACGCT | | Chr. 4 | |
| NO:15) | | GCCTACTGCTTGGGTCTAA | | | |
| 17. | (TTAGGG)₃₇ | AGAGAGAGAGGCACAGCAGGAGGTCTCTCAA | 60 | Chr. 17 | (CCCTAA)₄ |
| (SEQ ID | | CTCGACTGCGAGTCGCCGTCTCAGCCTT | | | |
| NO:16) | | | | | |
| 18. | (TTAGGG)₃ | AGGCTGAGGCAGGCGGATCACGAGGTCAGGA | 109 | Chr. 12 | (CCCTAA)₃₉ |
| (SEQ ID | | GATTGAGACCATCCTGGCTAACATGATGAAA | | | |
| NO:17) | | CCTCGTCTCTACTAAAATACAAAAATTACCT | | | |
| | | AATGTGGTGGTGG | | | |

| | |
|---|---|
| 19. | Both of the samples, 9 and 19, are harboring the inserts of same sequence |

| | | | | | |
|---|---|---|---|---|---|
| 20. | (TTAGGG)₅ | TAATCCCATCACTTTGAGAGGTCAAGGTGGG | 104 | Chr. 8 | (CCCTAA)₃ |
| (SEQ ID | | AGGACTCGTTGATCGACAGTGACCCAGTCTT | | | |
| NO:18) | | GAAAAAAGATACGGAAGACGACTGCCATGTG | | | |
| | | ACCTGTCT | | | |

TABLE 2
Class I Breast Tumor Tissue Fusion Junction (DCIS only)

| | | | | | | | | Known |
|---|---|---|---|---|---|---|---|---|
| Class | # of | | Length | Origin | | E- | Repeat | Fragile |
| I | Clones | Fusion Junction Sequence | (bp) | (CHr.) | Identity | Value | Type | Site |
| 1 | 2 | ATGATTACAA TTTCAATTTC TAACCTGTTT TATTTTGTTT | 109 | 6p25.3 | 100 | 1e−34 | Alu | FRA6B |
| SEQ | | TTTTTCTGAG ACAGGGTCTC CCTCTTTTGT CCAAGGCTGG | | | | | | |
| ID:46 | | AGTGTGGTAG CGTATCACAC GTGACTCGA | | | | | | |
| 2 | 1 | TGCAGTCAGC TGTGATAGCA CTACCACACT CCAGCCTTGG | 105 | 1q42.11 | 100 | 5e−46 | Alu | FRA1H |
| SEQ | | ACAACAGAGG GAGACCCTGT CTCAGAAAAA AAAACAAAAT | | | | | | |
| ID:47 | | AAAACAGGTT AGAAATTGTA TCTGC | | | | | | |
| 3 | 5 | TCAGGGTCAG GGTCAGGGTT AGGGTCAGGG TAAGGGTCAG | 664-832 | 5p15.33 | 100 | 1e−40 | n/a | FRA5B |
| SEQ | | GGTACAGCAC TTCCGGGTTA GGGTCAGGGT TAGGGTCAGG | | | | | | |
| ID:48 | | GTACAGCACT TCCGGGTTAG GGTCAGGGTT AGGGTCAGGG | | | | | | |
| | | TCAGGGTACA GCACTTCCGG GTTAGGGTCA GGGTCAGGGT | | | | | | |
| | | CAGGGTTAGG GTCAGGGTCA GGGTCAGGGT ACAGCACTTC | | | | | | |
| | | CGGGTCAGGG TCAGGGTCAG GGTCAGGGTC AGGGTTAGGG | | | | | | |
| | | TCAGGGTCAG GGTCAGGGTA CAGCATTTCC AGCGCTGCCA | | | | | | |
| | | GCGGCCTCCT TGCTTATGGT GCATGTTCTA ACCCTCACAA | | | | | | |
| | | ACCACAAGCT TGTCTTTAAA AACATCAAGT TGAAATAAAC | | | | | | |
| | | CACATATTAA TTGAGGTAAA ATAAGTGGCC AGAGAACCCA | | | | | | |
| | | CATAATTTAG TTGCAGTAAA CTTCTGCTGC ATATTTAAAG | | | | | | |
| | | GAAAATAAAC GAAATAATAG TTTTCAAAAC ATAAAAATTA | | | | | | |
| | | TTCCACTCTT TCTGAAAACA CACTGCTAAT CTAAGCCTAA | | | | | | |
| | | CCAAAAAGCT AACCCTAACC CTACCACTAA CCCTAATGCT | | | | | | |
| | | ACCACTAGCC TCTAACCCTA CTGCTAACTC TAACACCTAA | | | | | | |
| | | CCCTAATCCC AAACCCCTAA ACCAAACCCT AGTCCTGAAC | | | | | | |
| | | CCTAACCCTA ACCCCAACCC AAACCCCAAT ACCGACACTA | | | | | | |
| | | CACTAATCAT AACCCAACCC TAACTCTAAC CGCAAAACCC | | | | | | |
| | | TAACCCCTGA CCCTAAACCC AACCCCAACC CAAAACCTAA | | | | | | |
| | | CTCTAACCCC TGAACCAAAC CCTAAAACCC CACAAACCCC | | | | | | |
| | | AAAAATATTC CAAACCGAAC CTCACCCTAA CC | | | | | | |
| 4 (15) | 2 | TTTGGGTTAG AGTTAGAGTT AGAGTTAGAG TTAGAGTTAG | 424,592 | 10q11.22 | 100 | 1e−56 | n/a | FRA10G |
| SEQ | | AGTTAGAGTT AGGAATGTCA ACCCCATTGT CAGTGGCTGA | | | | | | |
| ID:49 | | CTCTCAGTTG CCCATCCCTG GTTCAGGGTT AAAGTTAGGG | | | | | | |
| | | TTTGGGTTAG AGTTAGAGTT AGAGTTAGAG TTAGAGTTAG | | | | | | |
| | | AGTTAGGAAT GTCAACCCCA TTGTCAGTGG CTGACTCTCA | | | | | | |
| | | GTTGCCCATC CCTGGTTCAG GGTTAGGGTT AGGGTTAGGT | | | | | | |
| | | TTAGGAAAGT CAGCCCCATT GCAGTTCCTG ACTCACAGTT | | | | | | |
| | | GCTCATCCAT GGGAAACTCC TACTGTCACC AGAGATGGTC | | | | | | |
| | | CAAGCAGGGC CCTGGTGAAG TTCCCCAGGC CTGCATTCTC | | | | | | |
| | | TGTAACTCGG ATGAGCTCAG AAGGGCTTGA AATCTCTGGT | | | | | | |
| | | CAAAATCACA ATGAGGAATG AGGAGGACAA AGCCCTTGCC | | | | | | |
| | | TGGGCCCCTC CTTCATCCAG GAGGACTGGC GCAAAGAACA | | | | | | |
| | | GTGGCTCCCG GAGAGCTTGG GAGCTGATTT TTAACAGTCA | | | | | | |
| | | ATGTCTTTCC AGGTCAACCA CCTTTTTAAA TTTTTTTCAG | | | | | | |
| | | GAATAGAATA AAAAACGGTC TTGACCAACT TC | | | | | | |

TABLE 3
Class II Breast Tumor Tissue (DCIS and advanced) Fusion Junction

| | | | | | | | | | |
|---|---|---|---|---|---|---|---|---|---|
| | | | | | | | | | Known |
| Class | # of | | | Length | Origin | | E- | Repeat | Fragile |
| II | Clones | Region | Fusion Junction Sequence | (bp) | (CHr.) | Identity | Value | Type | Site |

| | | | | | | | | | |
|---|---|---|---|---|---|---|---|---|---|
| 1 | 4 | A | TCAGGGTTAT GGTCAGGGTC AGGGTCAGGG | 174 | ?? | n/a | n/a | n/a | FRA16A |
| SEQ | | | TCAGGGTCAG GGTCAGGGTC AGGGTCAGGG | | | | | | |
| ID:50 | | | TTAGGGTTAG GGTCAGGGTC AGGGTCAGGG | | | | | | |
| | | | TCAGGGTCAG GGTCAGGGTC AGGGTCAGGG | | | | | | |
| | | | TCAGGGTCAG GGTCAGGGTT AGGGTCAGGG | | | | | | |
| | | | TCAGGGTCAG GGTCAGGGTC AGGG | | | | | | |
| SEQ | | B | TTTGTTACAT GGGTATATTG CATGATGCTA | 106 | 2p22.1 | 106/106 | 6e−52 | L1 | FRA2K |
| ID:51 | | | AGGTTTGGGG TATGACTGAT CCCATCACCC | | | (100%) | | | |
| | | | AGGTAGGGAC CATAGTATCC AATAGGTAGT | | | | | | |
| | | | TGTTCAGCCC TTTTCT | | | | | | |
| 2 | 21 | A | CACCCCAAGC AGTAGGCAGC GTCACCCCTA | 67 | 4p16.1 | 67/67 | 5e−29 | MIR | FRA4A |
| SEQ | | | TTTTGCAGGT AAAGACATGG AGGCTGAGTT | | | (100%) | | | |
| ID:52 | | | AAGTAAC | | | | | | |
| SEQ | | B | TC GCCCTCGCCC TCGCCCTCGC | 36 | 5q21 | 35/36 | 1e−08 | Other | FRA5F |
| ID:53 | | | CCTCGCCCTC GCCC | | | (97%) | | | |
| 3 | 8 | A | CTAGGGCTAG GGCTAGGGCT AGGGCTAGGG | 231 | ?? | n/a | n/a | n/a | FRA16A |
| SEQ | | | CTAGGGTTAG GGTCAGGGTC AGGGTCAGGG | | | | | | |
| ID:54 | | | TCAGGGTCAG GGTCAGGGTC AGGGTCAGGG | | | | | | |
| | | | TTTTAGGGTT AGGGTTAGGG TTAGGGTTAG | | | | | | |
| | | | GGTCAGGGTC AGGGTCAGGG TTAGGGTCAG | | | | | | |
| | | | GGTCAGGGTC AGGGTCAGGG TTAAGGGTCA | | | | | | |
| | | | GGGTCAGGGT CAGGGTCAGG GTCAGGGTCA | | | | | | |
| | | | GGGGTAGGGG TAGGGGTAGG | | | | | | |
| SEQ | | B | GCGCCCACCA CCACACTTGG CTAATTTTTT | 114 | 12q24.3 | 112/114 | 5e/52 | Alu | FRA12C |
| ID:55 | | | GTATTTTTAG TAGAGACGAG GTTTCATCAT | | | (98%) | | | |
| | | | GTTAGCCAGG ATGGTCCCAA TCTCCTGACC | | | | | | |
| | | | TCGTGATCCG CCTGCCTCAG CCTC | | | | | | |

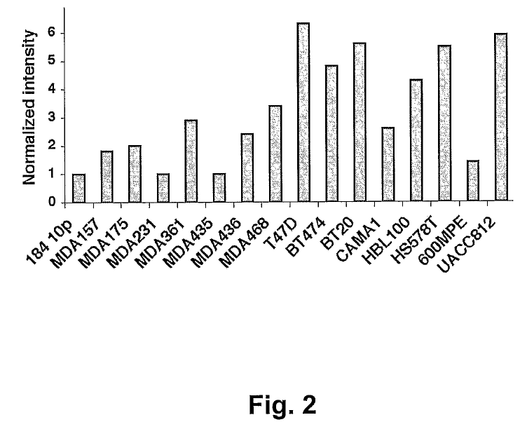

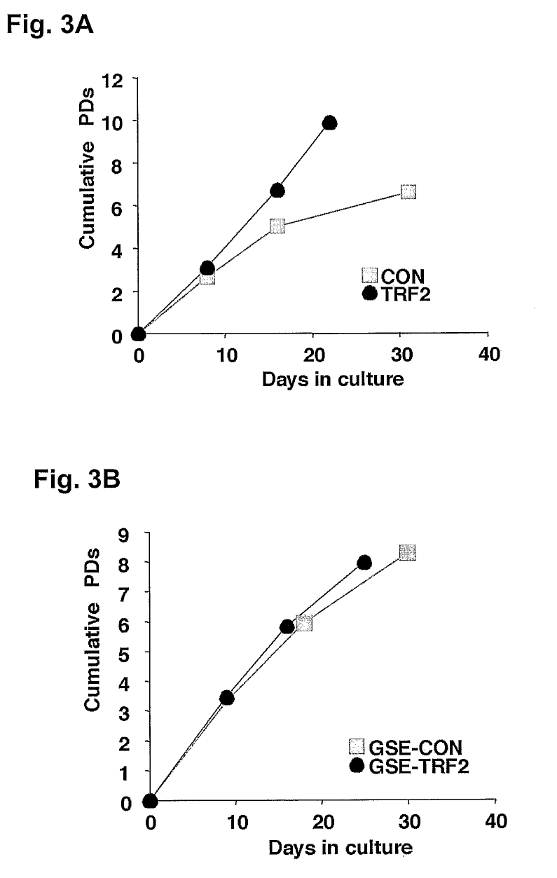

Another aspect of the present disclosure is directed to the discovery that immortalization of Human Mammary Epithelial Cells (HMECs), breast tumor-derived cell lines and breast tumor tissue have a dramatic upregulation (˜25-fold) of the telomere binding protein TRF2 (FIGS. 2D, 3A and 3B). TRF2 is a critical telomere capping protein, which binds directly to telomeric DNA and localizes to the t-loop junction (Griffith et al., (1999) Cell 97: 503-14).

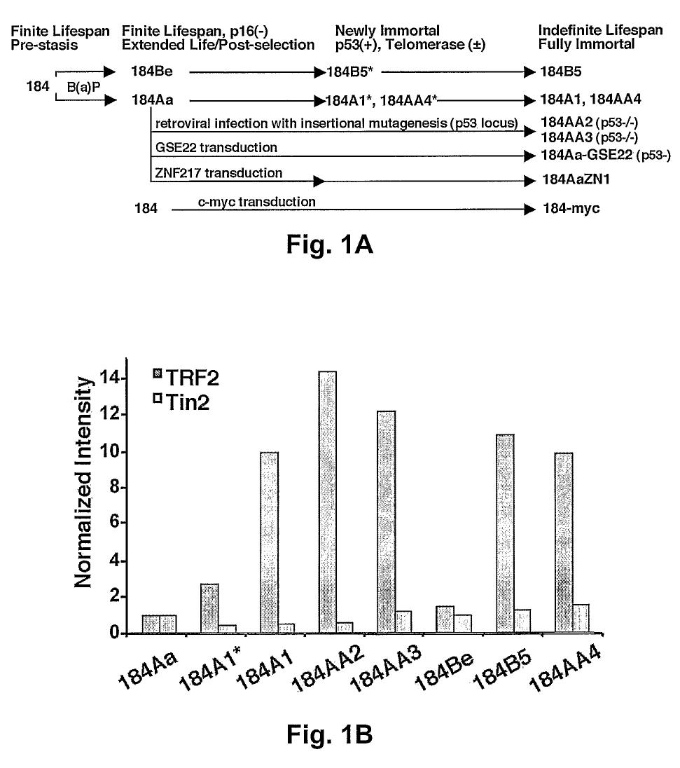

Protein lysates harvested from actively proliferating finite life span and immortal HMEC lines (see FIG. 1A) were examined for expression of telomere-associated proteins TRF2, TIN2, hRAP1, and TRF1. Immunoblots of total cellular protein probed with a specific monoclonal antibody to TRF2 indicated that four independently derived immortal HMEC lines (184A1, 184AA2, 184AA3, and 184AA4) displayed markedly increased levels of the 65/69 kD TRF2 doublet compared to their carcinogen-treated extended life precursor strain, 184Aa. A fifth immortal HMEC line, 184B5, derived from an independent carcinogen-treated extended life strain, 184Be, showed the same pattern. In contrast, levels of telomere-associated proteins, Tin2, hRap1, and TRF1 showed little differences in the same cultures.

Upregulation of TRF2 occurs at the post-transcriptional level with TRF2 mRNA levels remaining relatively comparable in both primary and immortalized HMECs. Dramatic TRF2 upregulation (25-fold increase or greater) was also seen in 67% (16 out of a total of 24) of tumor-derived cell lines tested. In summary, the results reported herein indicate a dramatic upregulation of TRF2 levels in immortalized HMECs, the majority of tumor-derived cell lines (17 of 24) and breast tumor tissue. Since TRF2 upregulation might be indicative of a possible compensatory mechanism to overcome an early event of telomere crisis, this is the first evidence that telomere dysfunction might occur in human breast cancers. Accordingly, the method of detecting TRF2 upregulation in breast tissue has significant clinical applications for early cancer detection. Thus, a further embodiment of the present disclosure relates to a method of detecting significantly elevated TRF2 in the cells of a tissue sample relative to normal cells, providing a diagnostic assay for the presence of cancerous and pre-cancerous cells in the tissue sample.

Comparison of TRF2 levels in common breast tumor cell lines with those in finite life span HMEC revealed a correlation between elevated TRF2 levels and cancer. Protein lysates were prepared from randomly cycling cells and analyzed by immunoblotting as described in Example 1. TRF2 levels were found to be at least 2-fold higher in breast tumor cell lines than in the control 184 cells in 11/15 lines examined, indicating that elevated TRF2 levels are a frequent occurrence in breast tumor cell lines (see FIG. 2).

In one embodiment a method of detecting aberrant TRK2 expression in a patient's tissue is provided. The method comprises contacting proteins of the patient's tissue with a labeled ligand that specifically binds to TRK2 (e.g. a TRK2 specific antibody), and then comparing the relative levels of detected TRK2 protein in the patient's tissues to that of a control sample representing “normal cells.” The proteins of the patient's tissue may be contacted either in situ, using thin slices of tissue, or the proteins can be first isolated from the cells and then contacted (e.g. by Western blot analysis). The control sample may represent normal cells isolated from a second sample taken from the patient, or may represent a sample (or mixture of multiple samples) obtained from another individual. More particularly, in accordance with one embodiment both the test tissue and the control tissue are taken from a similar source. In one embodiment the tissue is human female breast tissue. In one embodiment the amount of TRK2 detected in the test sample is compared to TRK2 levels detected in normal tissue to determine a diagnosis. Alternatively, the amount of TRK2 detected in the test sample can compared to TRK2 levels detected in known cancerous and precancerous tissue samples to determine a diagnosis. In another embodiment the amount of TRK2 detected in the test sample is compared to TRK2 levels in normal and cancerous cells prior to making a diagnosis. In one embodiment the comparison is conducted using standard immunoassay techniques using an antibody specific for TRK2. In one embodiment the comparison is made using Western blot analysis. Statistically significant elevated levels of TRK2 protein in the test sample relative to the control sample cells is indicative of a cancerous or pre-cancerous state.

Applicants have also observed that cancerous or pre-cancerous cells also display an alteration in the cellular localization of TRF2. Immunofluorescent studies of immortal HMEC, and tumor-derived cell lines with elevated TRF2, indicate that TRF2 is found throughout the nucleus, as opposed to the normal telomeric punctate pattern found in primary cells. Immunoflorescence co-localization studies of telomere proteins in tissue were conducted as follows. Cells were grown on 4-well chamber slides and fixed with 4% formalin. The slides were incubated with TRF2 and TIN2 specific antibodies at 5 μg/ml concentrations and then incubated with Texas Red conjugated anti-mouse IgG for TRF2 (red) and FITC conjugated anti-rabbit IgG antibodies for TIN2 (green). DNA was stained with DAPI (blue) in the merged images. Stained cells were visualized using an Olympus BX51 microscope equipped for epifluorescence. The immunoflorescence co-localization studies revealed that a critical portion of TRF2 staining (green) co-localizes with Tin2 staining (red) to form yellow punctate staining pattern typical of normal telomere protein staining. Essentially all Tin2 staining in tissue formed a typical telomere punctate pattern. In addition, the majority of the invasive cells display specific TRF2 staining that is not located at telomeres since it does not co-localize with Tin2. Therefore, the TRF2 and Tin2 staining in these studies are highly specific.

According, one aspect of the present invention is directed to methods of diagnosing cancerous and pre-cancerous cells based on the distribution of TRF2 present in the cells of a tissue sample relative to normal cells. In one embodiment the cells are isolated from a patient's tissue (e.g. breast tissue), and in one embodiment the cells are isolated from a patient biopsy sample. In one embodiment an immunoassay is conducted on thin sections of tissue prepared from a biopsy sample and compared to the staining produced on sections of normal breast tissue.

One embodiment of the present disclosure is also directed to antibodies that specifically bind to TRF2. In one embodiment the antibody is specific for a phosphorylated form of TRF2. In a further embodiment the antibody is a monoclonal antibody.

It is contemplated that any antibody or probe used in the present disclosure will be labeled with a “reporter molecule,” which provides a detectable signal. The label may include, but is not limited to fluorescent, enzymatic (e.g., ELISA, as well as enzyme-based histochemical assays), radioactive, and luminescent systems. It is not intended that the present invention be limited to any particular detection system or label.

In another embodiment a kit is provided for conducting telomere specific PCR amplification reactions of the present disclosure. Such a kit can be used to detect and analyze telomere fusions present in a biological sample. In accordance with one embodiment the kit comprises a PCR primer that specifically binds to and amplifies telomere fusion sequences. The kit can be further provided with instructional materials, additional reagents and disposable labware for conducting PCR amplifications.

In accordance with one embodiment the telomere specific PCR primer comprises the sequence TTAGGG (SEQ ID NO: 19), or multiples thereof, including for example (TTAGGG)₃ (SEQ ID NO: 20). In a further embodiment the PCR primer comprises the sequence (TTAGGG)ₙ (SEQ ID NO: 19, SEQ ID NO 42 and SEQ ID NO: 20) and a restriction endonuclease recognition sequence linked to the 5′ end of the (TTAGGG)ₙ sequence, wherein n is an integer selected from the range of 1 to 3. In one embodiment the PCR primer comprises a sequence represented by the general formula X-Y-(Z)ₙ, wherein X represents the sequence GGGNNN (SEQ ID NO: 44), Y represents a restriction endonuclease recognition sequence, Z represents the sequence of SEQ ID NO: 19; and n is an integer selected from the range of 1-6, or in another embodiment n is an integer selected from the range of 1-3. In one embodiment the kit is provided with a PCR primer consisting of SEQ ID NO: 21.

The reagents of the kit may include buffers and/or the polymerase enzyme. In one embodiment the kit is provided with thermostable polymerase such as the Taq polymerase, for example. In another embodiment the PCR primer provided with the kit is labeled, or reagents are provided for labeling the PCR primer or detecting the amplification product of the reaction. The detection reagents include, for example, DNA binding dyes and labeled probes that bind to telomere specific sequences such as the sequence of SEQ ID NO: 19. The nucleic acids and other reagents can be packaged in a variety of containers, e.g., vials, tubes, bottles, and the like. Other reagents can be included in separate containers and provided with the kit; e.g., positive control samples, negative control samples, buffers, etc.

### EXAMPLE 1

### Immortal HMEC Lines Exhibit Up-Regulation of TRF2 Protein

Total cell lysates were prepared from randomly cycling sub-confluent cultures of HMEC by lysing the cells with 2× SDS sample buffer. 50 μg of protein samples were resolved on polyacrylamide gradient gels and electroblotted to nylon membranes and probed with antibodies to TRF2 (IMG-124; Imgenex, San Diego, Calif. or SC-9143; Santa Cruz Biotech., Santa Cruz, Calif.) or TIN2 (gift of J. Campisi, LBNL). Gel loading equivalence and blotting efficiency were determined by staining the blots with Ponceau S (Helena Labs, Beaumont, Tex.) and/or probing with an antibody to beta-actin. The 184A1 cells were harvested at an early pre-conversion passage which shows low or negligible telomerase activity. Immunoblots of total cellular protein probed with the TRF2 antibody indicated that four independently derived immortal HMEC lines (184A1, 184AA2, 184AA3, and 184AA4) displayed markedly increased levels of the 65/69 kD TRF2 doublet compared to their carcinogen-treated extended life precursor strain, 184Aa.

Immunoblots also demonstrated an up-regulation of TRF2 protein in growth-arrested (GO), as well as actively cycling immortal 184A1, relative to finite life span post-selection 184 and carcinogen-treated extended life 184Aa HMEC. 184A1 14p cells are pre-conversion type cells and grow well, while 184A1 19p cells have begun the conversion process and show poor growth. Cells were growth-arrested as described (Stampfer et al., (1993). Exp. Cell Res., 208, 175-188). Relative levels of telomere-associated proteins TIN2 and RAP1 were analyzed using anti-hRAP (IMG-272; Imgenex), or anti-TIN2 antibodies. Immunoblots comparing relative levels of TRF2 protein in two finite life span post-selection (184 and 161), two carcinogen-treated extended life (184Be and 184Aa), and one fully immortal (184A1) HMEC, as well as two human breast tumor cell lines (MDA468, and T47D) were also conducted. TRF2 could be detected in lysates of the extended life cultures in longer exposures.

Northern blots were prepared using 10 μg of total RNA per sample as described previously (Nijjar et al., (1999). Cancer Res., 59, 5112-5118). The blots were hybridized to a ³²P-labeled, 1200-bp EcoR1:Xho1 TRF2 cDNA probe. The TRF2 signal was measured using a phosphoimager and quantitative comparisons of TRF2-specific signals were performed using the ImageQuant software program. The ratios of signal intensities for the main TRF2 transcript divided by that of the ethidium bromide stained 18S rRNA were measured. Northern blots were conducted comparing the relative levels of TRF2 mRNA in the HMEC described above as well as three human breast tumor cell lines (MDA436, MDA468, and Hs578T). The Northern blot data indicated that upregulation of TRF2 occurs at the post-transcriptional level with TRF2 mRNA levels remaining relatively comparable in both primary and immortalized HMECs.

Dramatic TRF2 upregulation (25-fold increase or greater) was also seen in 67% (16 out of a total of 24) of tumor-derived cell lines tested. In summary, the results reported herein indicate a dramatic upregulation of TRF2 levels in immortalized HMECs and the majority of tumor-derived cell lines (17 of 24). In addition, TRF2 levels were also observed to be high in breast tumor derived cell lines. Protein samples isolated from breast tumor derived cell lines were analyzed by immunoblotting as described above for the tumor-derived cell lines. Quantification of the immunoblot data is shown in FIG. 2. Signal intensities for TRF2 bands were divided by those for the control bands, and plotted relative to the levels in 184.

### EXAMPLE 2

Accumulation and Altered Localization of Telomere-Associated Protein TRF2 in Immortally Transformed and Tumor-Derived Human Breast Cells Telomeres, the nucleoprotein structures that cap the ends of the eukaryotic chromosomes, are critical for chromosomal integrity. Composed of TTAGGG (SEQ ID NO: 19) DNA repeats bound by a complex of proteins, these specialized structures protect the chromosome ends from exonucleolytic attack and fusion. Due to the “end replication problem,” in the absence of telomerase a specialized enzyme that maintains telomeric DNA, telomeres are eroded with successive cell divisions. When telomeres become critically eroded, the ensuing telomere dysfunction can produce genomic instability, resulting in growth suppression or cell death. Under normal circumstances, the finite life span conferred by telomerase repression in human cells limits the number of mutations that can accumulate in a single cell lineage, and serves as a stringent block to tumorigenesis.

Binding of TRF1 and TRF2 proteins and their interacting partners to the telomeric repeats is thought to reorganize the linear chromosome terminus into a protective t-loop structure, in which the G strand invades the duplex part of the telomere. TRF2 binding near the 3′-overhang is considered crucial to the formation and stability of t-loops. Interference with TRF2 function by over-expression of a dominant negative form of TRF2 results in telomere dysfunction, genomic instability, and a proliferative growth arrest with features characteristic of senescence. On the other hand, artificially over-expressed TRF2 has been reported to delay senescence.

HMEC cultured from normal breast tissue display a finite life span, low or undetectable telomerase activity, and decreasing telomere length with passage (Stampfer & Yaswen, (2003) Cancer Lett, 194, 199-208). HMEC can spontaneously overcome a first RB-mediated, non-telomere length dependent proliferative arrest (stasis), associated with down-regulation of p16 expression. The resultant p53(+), p16(−) post-selection HMEC cease net proliferation when their mean terminal restriction fragment (TRF) length is ˜5 kb. As cells approach this second proliferative barrier, telomere dysfunction is evidenced by the presence of widespread chromosomal aberrations, particularly telomeric fusions, and mitotic failures. In the p53(+) cultures, most cells remain viably arrested at all phases of the cell cycle, a growth arrest termed agonescence. When p53 is inactivated, populations display the massive cell death typical of crisis. Rare p53(+) and p53(−) immortal HMEC lines have been obtained following exposure to chemical carcinogens, over-expression of c-myc or ZNF21 7 oncogenes, and/or a dominant negative p53 genetic suppressor element, GSE22 (FIG. 1A). Surprisingly, the newly immortal p53(+) lines initially show very low or undetectable telomerase activity and continue to divide with increasingly shortened mean TRF lengths. When the mean TRF length gets extremely short (<3 kb), growth becomes slow and heterogeneous. An extended process, termed conversion, ensues, during which telomerase activity and growth capacity gradually increase. In contrast, newly immortal p53(−) lines quickly display telomerase activity (Stampfer & Yaswen, (2003) Cancer Lett, 194, 199-208).

Recent studies have indicated that p53 is able to repress the expression of endogenous hTERT, the catalytic subunit of telomerase, in newly immortal lines; this repression is relieved during the process of conversion. Although telomerase activity remains very low until conversion, this low activity may be responsible for the observation that, unlike cells at agonescence, newly immortal p53(+) lines can continue to divide without exhibiting gross chromosomal instability.

TRF2 Protein Levels Undergo Large Increases in Immortally Transformed HMEC

Protein lysates harvested from actively proliferating finite life span and immortal HMEC lines were examined for expression of telomere-associated proteins TRF2, TIN2, hRAP1, and TRF1. The asterisk associated with 184A1* indicates that these cells were harvested at an early pre-conversion passage which shows low or negligible telomerase activity. Immunoblots of total cellular protein probed with a specific monoclonal antibody to TRF2 indicated that four independently derived immortal HMEC lines (184A1, 184AA2, 184AA3, and 184AA4) displayed markedly increased levels of the 65/69 kD TRF2 doublet compared to their carcinogen-treated extended life precursor strain, 184Aa (FIG. 1B). A fifth immortal HMEC line, 184B5, derived from an independent carcinogen-treated extended life strain, 184Be, showed the same pattern. In contrast, levels of telomere-associated proteins, Tin2, hRap1, and TRF1 showed little differences in the same cultures. The normalized levels of TRF2 protein observed in the immortal lines ranged from 10-15 times the levels present in the 184Aa precursor strain.

Interestingly, the newly immortal, pre-conversion 184A1 line, with low or undetectable telomerase activity, displayed intermediate levels of TRF2 protein. The TRF2 levels were higher in the immortalized cells regardless of whether the cells were actively cycling or growth arrested in G0 by blockage of EGFR signal transduction. Levels of TRF2 protein were approximately equivalent in independently derived finite life span strains 184 and 161, and in extended life strains 184Be and 184Aa. A second TRF2 polyclonal antibody yielded identical results.

DNA damage induced by irradiation and etoposide has been reported to induce the temporary accumulation of TRF2 mRNA in human promyelocytic HL60 cells. To determine whether the increased levels of TRF2 protein observed in immortalized HMEC correlated with increased TRF2 transcript levels, total RNA from growing HMEC cultures was subjected to northern blot analysis. Unlike the large differences detected in TRF2 protein levels, differences in TRF2 mRNA levels were fairly small and did not correlate with the differences in protein levels. The lack of correspondence between mRNA and protein differences suggests that variations in post-transcriptional regulation of TRF2 protein abundance exist among finite life span and immortalized HMEC. Inhibition of de novo protein synthesis using cycloheximide indicated that the half-life of TRF2 protein was greater than 12 hours in both finite life span 184 and fully immortal 184A1 HMEC. Although this experiment did not rule out differences in protein stability, it indicated that TRF2 levels are not regulated by rapid turnover, even under normal conditions. Thus, the difference in TRF2 protein accumulation is unlikely to be due to a simple change in stability, and is more likely to be due to changes in synthesis, modification, and/or compartmentalization.

Immortalizing Factors or Telomere Dysfunction do not by Themselves Directly Affect TRF2 Levels

To determine whether treatment with immortalizing factors by themselves was sufficient to cause up-regulation of TRF2, the following experiment was conducted. Finite life span cells treated with four different immortalizing agents (the chemical carcinogen benzo(a)pyrene±retroviral introduction of the dominant negative inhibitor of p53 function, GSE22 [Ossovskaya et al., (1996). Proc. Natl. Acad. Sci. USA, 93, 10309-10314], the c-myc oncogene, or the ZNF217 oncogene) were compared with the immortal cell lines derived from these cultures following exposure to these agents. Immunoblots were conducted to determine the total TRF2 protein in post-selection in 184, carcinogen-treated extended life 184Aa, and immortalized HMEC, after transduction with dominant negative p53 genetic suppressor element (GSE-22), oncogene c-myc, or oncogene ZNF217.

In all cases, the low level of TRF2 expression seen in unexposed finite life span HMEC was not significantly increased in the finite life span cultures that had been exposed to these agents. In contrast, TRF2 protein levels were increased in the resulting immortally transformed lines. The level of TRF2 was also not significantly increased in cultures when they reached agonescence. Since agonescence is associated with telomere dysfunction, end-to-end fusions, and genomic instability, these results indicate that TRF2 protein levels in HMEC are not stably elevated in response to telomere dysfunction alone.

Expression of Exogenously Introduced hTERT does not Lead to Increased TRF2 Levels

During its conversion to full immortality, the immortal 184A1 line displayed slowly increasing expression of endogenous hTERT and telomerase activity (Stampfer et al. (2003) Oncogene, 22, 5238-5251). Transduction of exogenous hTERT into post-selection finite life span 184 HMEC or immortal 184A1 (before, during, and after conversion) produced rapid elevation of telomerase activity, telomere elongation, and acquisition of an indefinite life span (Stampfer et al., (2001) Natl. Acad. Sci, USA., 98, 4498-4503). To determine directly whether increased TRF2 expression might be a consequence of the expression of hTERT, the hTERT-transduced 184 and 184A1 cultures were assayed for TRF2 expression. TRF2 levels were not increased in 184 HMEC immortalized by hTERT transduction. TRF2 levels also remained low in the early passage 184A1 line transduced with hTERT prior to conversion, a manipulation that circumvents the slow, heterogeneous growth phase associated with conversion. TRF2 levels in 184A1 cells transduced with hTERT during or after conversion to the fully immortal phenotype were consistent with the levels present at the time of transduction, and did not appear to be affected by the presence of added hTERT. Thus, over-expression of exogenously introduced hTERT did not influence TRF2 protein levels.

TRF2 Levels are Elevated in many Breast Tumor-Derived Cell Lines

To correlate the relevance of TRF2 elevation to human breast cancer, TRF2 levels in common breast tumor cell lines were compared with those in finite life span HMEC (FIG. 2). Protein lysates were prepared from randomly cycling cells and analyzed by immunoblotting. TRF2 levels were found to be at least 2-fold higher in breast tumor cell lines than in the control 184 cells in 11 out of 15 lines examined, indicating that elevated TRF2 levels are a frequent occurrence in breast tumor cell lines. Levels of TRF2 protein in these tumor lines did not correlate with the relative levels of mRNA from the same lines.

Exogenously Introduced TRF2 Affects the Proliferative Life Span of Post-Selection HMEC

When artificially over-expressed in human diploid fibroblasts, TRF2 has been reported to bind ATM kinase and repress cellular responses to genome-wide DNA damage (Karlseder et al., (2004) PLoS Biol, 2, E240). To directly determine the consequences of increased TRF2 expression for growth and immortalization of post-selection HMEC, additional copies of the TRF2 gene under control of the CMV promoter were retrovirally introduced into finite life span 184 HMEC alone, or with the dominant negative p53 element, GSE22.

To subclone TRF2 into the retroviral vector pBabe (Morgenstern & Land, (1990) Nucl. Acids Res., 18, 3587-3596), for LTR driven expression, a 1500 bp cDNA fragment encompassing the entire open reading frame was excised with BamH1:EcoR1 from the pLPC.TRF2 vector and subcloned into the BamH1-EcoR1 site of pBabe.Pu. The derivation of other retroviruses has been described. Post-selection 184 HMEC were transduced with retroviruses encoding TRF2 or empty vector (CON) alone or with a dominant negative inhibitor of p53 function (GSE) and selected in 0.5 μg/ml puromycin. The transduced cells were grown in the presence of selective drugs to confluence, then replated in triplicate at a fixed density of 1×10⁵/60 mm dish. The total number of cells harvested at every subculture was calculated and the number of accumulated population doublings (PD) per passage determined using the equation, PD=(A/B)/log2, where A is the number of harvested cells, and B is the number of plated cells, not corrected for plating efficiency. Experiments were terminated when the cultures failed to achieve confluence within 3 weeks. Each experiment was repeated three times and in each case representative data from one experiment is shown.

High expression levels of TRF2 protein were confirmed by immunoblotting. The number of cumulative population doublings (PD) prior to agonescence was modestly increased compared to controls in post-selection 184 transduced with TRF2 (FIG. 3A), similar to results previously reported for human diploid fibroblasts (Karlseder et al., 2002) Science, 295, 2446-9. Similar results were also obtained using a second vector in which TRF2 expression was driven by a retroviral LTR instead of the CMV promoter in order to achieve lower TRF2 levels more consistent with endogenous levels observed in immortal cell lines.

Since TRF2 is a crucial stabilizing component of the protective t-loop structure, up-regulated TRF2 may provide increased stability when the telomeres are relatively short. Over-expressed TRF2 may postpone telomere dysfunction by providing added protection to the telomeric ends (i.e., additional telomere erosion may be required to produce telomere dysfunction and the signal for p53-dependent growth arrest). In contrast, transduction of 184-GSE22 cells with TRF2 did not affect the number of cumulative PD achieved (FIG. 3B). The inability of TRF2 over-expression to further increase the cumulative PD in cells with compromised p53 was also reported in fibroblasts (Karlseder et al., 2002) Science, 295, 2446-9. This data suggests that the increased telomere protection conferred by over-expressed TRF2 is short-lived, and does not interfere with p53-independent events that ultimately result in crisis.

TRF2 Localization is Abnormal in Immortal HMEC and Breast Tumor Cell Lines

Indirect immunofluorescent studies with the anti-TRF2 antibodies revealed a punctate nuclear pattern in all interphase post-selection 184 HMEC, similar to that first reported in HeLa cells. However, in immortal HMEC, TRF2 immunofluorescence was heterogeneous both in abundance and localization. Both 184A1 and 184AA2 HMEC displayed gradations in nuclear size and TRF2 protein expression levels. Cells with smaller nuclei showed quantities and punctate distributions of TRF2 similar to those found in finite life span cells, where TRF2 co-localized with Tin2. However cells with larger nuclei had correspondingly high levels of TRF2 spread throughout the nuclei (although a portion of TRF2 remained co-localized with Tin2). A gradient of cells with intermediate characteristics was also observed. Tin2 levels and localization were similar in finite life span and immortal HMEC regardless of nuclei sizes or differences in TRF2 levels and localization. Tumor cell lines, T47D and BT474, with high levels of TRF2 on immunoblots, uniformly displayed TRF2 dispersed throughout the nuclei in essentially all cells, indicating a lack of dependence on cell cycle status. A contrasting tumor cell line MDA435, which displayed low levels of TRF2 by immunoblotting, uniformly displayed TRF2 in the punctate pattern typical of finite life span HMEC. Both TRF2 antibodies used in these studies yielded essentially the same results.

TRF2 Protein Abundance is Increased in some Aberrant Breast Tissues

Initial immunohistochemical experiments were performed using sectioned formalin-fixed, paraffin-embedded human breast tissues provided by the UCSF Cancer Center Tissue Core and the Breast Oncology Program. These experiments, performed with two different monoclonal anti-TRF2 antibody preparations, showed obvious positive staining in epithelial cell nuclei in some areas of DCIS and invasive breast cancers. Staining of stromal and normal ductal epithelial cells in these sections was noticeably weaker.

The mechanism responsible for the up-regulation of endogenous TRF2 in immortalized HMEC remains to be determined, but may involve altered post-translational modifications and/or protein interactions since mRNA levels are unaffected. Blackburn ((2000). Nature, 408, 53-56) proposed a model in which telomeres exist in two interchangeable states, an open accessible form and a closed protected form. Evidence suggests that TRF2 binding to TTAGGG (SEQ ID NO: 19) repeats promotes formation of the closed protected form. When eroded telomeres become critically shortened, the ends on one or more chromosomes may lack sufficient TTAGGG (SEQ ID NO: 19) repeats to stably bind TRF2, thereby limiting formation of the protective t-loop structure and allowing loss/degradation of the 3′ overhang. Proteins involved in DNA double strand break recognition and repair may participate in the cellular response to persistent unprotected telomeric structures. Normally, such structures may be resolved by progression of the associated repair pathways, including DNA ligase IV-dependent non-homologous end-joining of unprotected telomeres.

TRF2 has been reported to bind to several proteins involved in double strand break recognition and repair, including the Rad50-MRE11-NBS1 complex, ATM, as well as the RecQ helicases—WRN and BLM. These proteins may respond to particular telomeric structures by interacting with and stabilizing or destabilizing TRF2 protein. However, in some cases, it is possible that molecular defects inhibit the resolution of the intermediates, causing accumulation of TRF2 protein. Alternatively, molecular defects in HMEC undergoing immortalization may cause up-regulation of TRF2 protein independently of telomere dysfunction. The dispersed distribution of over-expressed TRF2 throughout the nuclei in some immortalized and tumor-derived cells indicates that not all the TRF2 is associated with telomeres in these cells.

### EXAMPLE 3

### Use of Telomere Fusions as Diagnostic Markers of Cancer

Telomere dysfunction is one of the key driving forces behind the genomic instability observed in early breast lesions. As proposed by applicants, telomere capping is believed to be disrupted in a small subset of normal breast epithelial cells. This loss of telomere function then results in telomere fusions, causing genomic instability via breakage-fusion-bridge cycles during subsequent cell cycles. Telomere dysfunction is likely indicated by alterations in telomere-associated protein levels, disruption of critical telomere-associated protein-protein interactions, deregulation of telomere-associated protein modification (e.g., phosphorylation), loss of a critical tissue-specific telomeric DNA length, and by the accumulation of telomere fusions. Consequently, loss of genomic integrity leads to misregulation of genes involved in growth control (including hTERT—the catalytic component of telomerase), ultimately resulting in tumorigenesis.

Several recent reports support the theory that defects in telomere maintenance initiate genomic instability eventually resulting in the development of breast cancer and other cancers. However, the extent of telomere dysfunction in human breast cancer (and other cancers) has not been directly determined due to present methodological limitations in detecting telomere dysfunction in tissue. Telomere length can be readily determined in tissue, however, telomere shortening does not necessarily indicate loss of telomere function, and telomere dysfunction can occur without telomeric DNA shortening. Accordingly, the present disclosure provides reagents and methods for examining loss of telomere function during breast tumorigenesis.

To overcome current methodological limitations and to directly determining the extent of telomere dysfunction during breast tumorigenesis, two innovative assays were developed that: 1) detect telomere fusion in cell lines and tissue, and 2) localize telomere-associated proteins in tissue sections.

### Materials and Methods

### Cell Cultures.

During these studies, two lines of BJ foreskin fibroblasts were used: BJ and BJ HPV E6/E7 cells. BJ cells have a population doubling (PD) of 50 and represent a control primary cell line that does not contain detectable levels of telomere fusions. BJ HPV E6/E7 cells have a PD of 85 and contain significant numbers of cells with telomeric fusions, about 35% as determined by metaphase spreads. Genomic DNA was isolated from each cell and digested with tetra cutter restriction enzymes Rsa I and Hinf I, to free sample preparations of non-telomeric DNA for use as template for PCR reaction. In addition, finite life span human mammary epithelial cells were used that do not contain (early passage) and do contain (late passage) telomere fusions.

### PCR Primer & Reaction Condition.

The sequence of the PCR fusion junction primer is 5′-GGGNNNGAATTC(TTAGGG)₃-3′ (SEQ ID NO: 21). Maintenance of the 5′ to 3′ DNA strand at the ends of the fusion junction between telomere-telomere associations should allow for the amplification of the telomere-telomere junction through the use of this one primer via PCR (see FIG. 5). The results reported herein confirm this to be true, and conditions that amplify specific products from BJ fibroblasts that contain telomere fusions were determined. In order to facilitate the cloning of the PCR product, an EcoR I site was introduced at the 5′ end of the primer. The reaction conditions used were as follows: initial denaturation (2 min at 94° C.), 32-35 cycles, (30 s 94° C., 1 min annealing at 63° C., 2 min 72° C.) and final extension step (5 min, 72° C.).

### Southern Blotting.

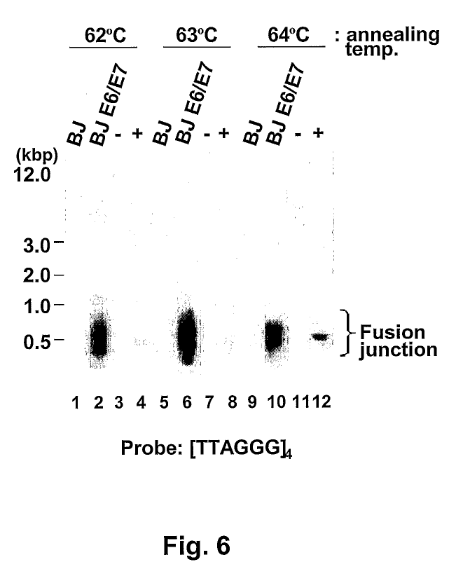

To determine whether specific telomeric chromosomal regions were amplified, PCR products were run on 0.8% agarose gels and blotted on Hybond-N+membrane (Amersham) and hybridized at 42° C. for 12 h with [TTAGGG]4 probe (SEQ ID NO: 43; labeled using a kinase reaction with [gamma ³²P]ATP). A phosphor screen was exposed to the membrane for 3 h and images were detected by a phosphoimager (Amersham). An example is shown in FIG. 6, in which telomeric products are seen only with the BJ fibroblasts that contain fusion but not in the earlier passage of BJ fibroblasts that do not contain telomere fusions.

### Cloning & Sequencing.

The PCR product were digested with EcoR I and cloned in EcoR I cut pBluscript plasmid. The recombinant plasmids were screened by colony hybridization to pick clones that specifically contain telomeric DNA. Sequencing of the insert was performed by an automated DNA sequencer (see attached Table 1 for examples of telomere fusion sequences).

### Detection of Telomere Fusion Using Breast Tumor Tissue.

DNA will be isolated from normal and breast tumor to perform PCR with the fusion junction primer using the same method used for BJ fibroblast. PCR conditions will be developed to specifically amplify telomere fusions from breast tumor tissue. Late passage BJ fibroblasts and HMECs that contain telomere fusions, along with early passage cells that do not contain fusion as a negative control, will be used to make metaphase spreads and whole cells fixed on chamber slides for in situ PCR amplification of fused telomere-telomere associated chromosomes junctions. Metaphase spreads and whole cells will be prepared on a chamber slides for in situ PCR using a FITC-labeled “fusion primer” adapted. It is anticipated that breast tumor tissue sections will ultimately be used to determine whether fusions occur in the samples.

One hypothesis implicates telomere dysfunction as a key driving force behind the genomic instability observed in early malignant lesions (see FIG. 4). The telomere dysfunction hypothesis states that telomere capping is disrupted in a small subset of normal precursor cells. This loss of telomere capping then results in telomere fusions causing genomic instability via breakage-fusion-bridge cycles (FIGS. 4 and 8). Consequently, loss of genomic integrity leads to misregulation of genes involved in growth control (including TERT—the catalytic component of telomerase), ultimately resulting in tumorigenesis. Telomere dysfunction can be induced by aberrant telomeric DNA length and/or loss of function of a critical telomere-associated protein, even without changes in telomeric DNA length.

Therefore, although telomere shortening is likely an important cause of telomere dysfunction, it may not be the only cause of telomere dysfunction in cancers. Defects in the regulation and modification of telomere-associated proteins may also play an important role in telomere dysfunction in cancers. When telomere dysfunction was induced experimentally by a deficiency in the telomerase RNA component (mTER) in a p53 mutant mouse background, high levels of breast adenocarcinomas and other epithelial cancers were observed that do not normally occur in these strains (Artandi et al., (2000) Nature Aug 10;406(6796):641-5). In addition, they discovered that the constitutive upregulation of telomerase in transgenic mice was associated with the spontaneous development of mammary carcinomas, questioning the idea that telomerase expression can be safely used to immortalize human cells for therapeutic purposes without an increase risk for malignancy.

### Telomere Fusion Analysis and Detection.

To test the telomere dysfunction hypothesis directly, a unique PCR-based method was developed to detect, clone and sequence end-to-end telomeric fusions in mammalian cells. The accumulation of telomere fusions is a definitive hallmark of telomere dysfunction, and thus the PCR-based methodology disclosed herein provides a unique analytical and diagnostic tool for cancer related applications. Additionally, this analysis has provided important clues into possible mechanisms of these specific breakage-fusion-bridge cycles.

Two lines of BJ foreskin fibroblasts—BJ and BJ HPV E6/E7 were initially used to conduct these studies. BJ cells have a population doubling (PD) of 50 and represent a control primary cell line that does not contain detectable levels of telomere fusions. BJ HPV E6/E7 cells have a PD of 85 and contain significant numbers of cells with telomeric fusions, about 35% as determined by metaphase spreads. Through systematic preliminary studies, specific primer sequences were determined empirically that eliminated non-specific genomic background signal. The principle of the PCR method for detecting and cloning telomere fusion junctions is shown in FIG. 5). Detail of the fusion junction between two chromosomal end-to-end fusions is shown with the PCR primer for the amplification of the telomere fusion junction.

Specific PCR conditions that amplify products only from BJ HPV E6/E7 fibroblasts containing telomere fusions were determined and the amplified fragments were detected by Southern hybridization (see FIG. 6). A strong diffuse signal is seen in lanes 2, 6 and 10 in FIG. 6 exclusively with the BJ HPV E6/E7 line (telomere fusion positive line). Presently, we have cloned and determined the sequence of approximately 30 telomere fusion junctions from BJ HPV E6/E7 cell line.

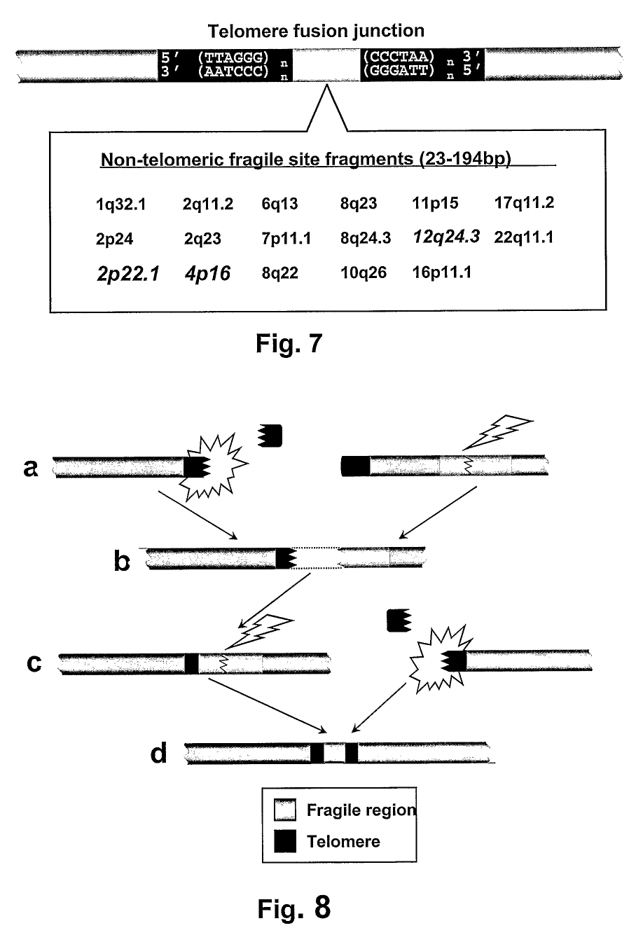

Amplified DNA was cloned and colonies were analyzed via colony hybridization technique using a telomere probe. Cloned fusion junctions were isolated and sequenced. The clones contained various sizes of telomeric repeats, and interestingly, fragments of non-telomeric DNA (23-194 bp) are inserted between telomere-telomere fusions in all clones examined to date (FIG. 7). Importantly, the non-telomeric DNA found inserted at telomere fusion sites, in 37 out of 40 clones sequenced, corresponds to previously identified fragile chromosome sites, thus supporting the concept that these are bona fide fusion junctions occurring in vivo, not PCR and/or cloning artifacts. Unexpectedly, we have not found telomere-telomere direct junctions after examining over 40 fusion junctions. Additionally, this analysis shows that the G-rich strand joins covalently to the C-rich strand of the joining chromosome maintaining a 5′ to 3′ DNA strand directionality and suggests that multiple breakage-fusion-bridge cycles take place to create telomere fusions (see the proposed working model presented in FIG. 8).

The fusion junction sequences of finite life span human mammary epithelial cells that do not contain (early passage) or do contain (late passage) telomere fusions are also being investigated. 6 clones have been sequenced from late passage HMECs that contain telomere fusions, and the fusion junctions found in HMEC also contain the identical fragile site fragment DNA within the telomere fusion junction (FIG. 7). 5 telomere fusions have also been cloned and sequenced from one breast tumor tissue sample (ductal and lobular carcinoma), strongly suggesting that telomere dysfunction does indeed occur during breast tumorigenesis. Two of these clones contain a 104 nt. fragment from fragile site 4p16 and three of the clones contain a 113 nt. fragment from fragile site 12q24.3 (FIG. 7). Remarkably, these are the same fragile site fragments found in cell culture lines known to contain telomere fusions (BJ E6/E7 and immortalized HMEC) (FIG. 7). Importantly, corresponding normal breast tissue did not contain telomere fusion as determined by the presently disclosed method.

Detection and Analysis of Telomere Fusions using Breast Tumor Tissue.

Breast tumor tissues of various stages of breast cancer have been collected with matching normal tissues from the Indiana University Tissue Procurement Core Facility. Approximately 60 breast tissue samples of each of the following types will be tested for telomere fusions: 1) normal tissue with no cancer diagnosis, 2) matching normal tissue with cancer diagnosis, 3) usual ductal hyperplasia (UDH), 4) atypical ductal hyperplasia (ADH), 5) fibroadenoma, 6) lobular in situ carcinoma (LCIS), 7) ductal in situ carcinoma (DCIS), 8) invasive ductal carcinoma, 9) invasive metaplastic carcinoma, and 10) lobular and ductal carcinoma. From each of the tissue samples, a portion of tissue (˜25 mg) will be aliquoted to isolate genomic DNA using DNeasy Tissue DNA purification kit (Qiagen). DNA samples from tissue will be used to perform PCR as described above with fibroblast and HMECs and Preliminary Studies Section C.4. PCR conditions will be optimized to specifically amplify telomere fusions from breast tumor tissue.

Telomere Fusion Detection using a Non-PCR Based Method.

An alternative, non-PCR-based approach will be used to confirm the results from the above PCR-based amplification method. The information previously obtained regarding the sequence within the telomere fusion junction can be utilize to generate probes for analyses of the fused chromosomes. Most potential and common fragile sites that are identified in this study will be used as probes. Genomic DNA will be isolated from target cells/breast tumor tissues and will be digested with at least five selected restriction enzymes that have no cutting site inside the probe. Approximately 30 μg of restriction-digested DNA from each sample will be loaded on an 0.8% agarose gel, transferred to a membrane and hybridized with individual high specific activity probes consisting of fragile site fragment sequence found within telomere fusion junctions (e.g., 12q24 and 4p16 fragments).

### A Real Time PCR Approach to Quantitate Relative Telomere Fusion Accumulation.

A semi-quantitative real time PCR approach will be used to measure telomere fusion rates between different tissue samples, using the same telomere primer as previously described (5′GGGNNNGAATTC(TTAGGG)₃-3′ (SEQ ID NO: 21)). The intercalating fluorescent dye SYBR green will be used to quantify dsDNA accumulation. A positive control standard that mimics telomere fusions with 15 telomeric repeats at the left and right fusion junction with a modified multi-cloning site of pBluescript (115 bp) at the telomere-to-telomere junction will be constructed. In addition, negative controls (e.g., linear pGEM-T-Easy vector harboring 11 telomeric repeats that does not contain fused telomeric DNA) will be used to determine specific PCR conditions. Initially, we will optimize the PCR conditions (concentrations of templates, primer, and probe; annealing temperature; and others) using the positive and negative controls. Control DNAs will be mixed with genomic DNA preparations from cell lines with known percentages of fusions and breast tumor tissue. Standard curves will be constructed using genomic preparations with known percentages of fusion versus Cₜ (Cₜ=the cycle threshold number, which is an arbitrary number of PCR cycles in which all of the PCR amplification graphs will be in the linear range). The Cₜ values from the sample will be extra plotted and the number of fusions will be determined

Localizing Telomere Fusions in Breast Tumor Tissue Sections using in Situ PCR.

Analysis of isolated genomic DNA for telomere fusions is an important method that will continue to give highly relevant information regarding telomere dysfunction. However, such analysis cannot give information regarding the specific cell types that contain telomere dysfunction (i.e., telomere fusions) within complex, heterologous breast tissue, and that ability to calculate telomere fusion frequencies in heterologous tissue samples will be difficult. Additionally, telomere fusions may occur relatively rarely in breast tumorigenesis. Therefore, it is possible that analysis of thin tissue sections may result in false negatives. To address these possibilities, a new method of in situ RT-PCR in whole mount/thick section tissues will be adapted to an in situ PCR method.

PCR conditions have been worked out in the laboratory to specifically amplify the telomere fusion junction DNA delineated by fusion junction primer 5′-GGGNNNGAATTC(TTAGGG)₃-3′ (SEQ ID NO: 21). The primer will be labeled with Cy5 and used in the in situ PCR method described below. Initially, in situ PCR will be used to detect the telomere fusions in both BJ E6/E7 and HMECs passages that contain known percentages of fusions as determined by metaphase spread/FISH analysis. Under specific conditions, positive nuclear fluorescent spots should be detected (using this in situ PCR procedure) at the same percentage as telomere fusion rates determined using the standard methods of chromosome metaphase spreads and FISH analysis. For example, a cell line with a 30% telomere fusion rate as determined by metaphase/FISH analysis should display the same percentage of nuclear spots (30%) by the in situ PCR fusion method. BJ (PD 50) and primary early passage HMECs will serve as critical negative controls for non-specific PCR amplification. The same methodology will be adapted for detection of the telomere fusions in breast tumor tissues.

Primers specific to telomere fusion junctions were designed and various conditions for successful amplification by these primer sets have been established by doing solution based PCR assays. The primers were tagged with Cy5 at 5′ end by the manufacturer at the time of synthesis (Applied Biosystems). Specificity of the primers for amplifying the region encoding the telomere fusion junctions has been determined by direct sequencing of the amplicons. BJ foreskin fibroblast cell lines, HMECs or breast tumor tissue samples will be fixed in 4% parafomialdehyde-15% sucrose solution overnight at 4° C. and stored at −80° C. till processed for in situ PCR. The slides will be incubated for 10 seconds at 105°-110° C. Optimal conditions for protease digestion of paraformaldehyde fixed biopsies/cell spreads will be determined. Insufficient digestion with Proteinase K will not permit enzymes access to the genomic DNA. Proteinase K digestion conditions will be standardized by performing digestions in graded concentrations of the enzyme for a fixed time at 37° C. The highest concentration of the enzyme that will not change the cytoarchitecture of the cells will be used. Appearance of salt and pepper dots is an indication of optimal digestion. At that stage the digestion will be stopped by incubating the slides for 2 minutes at 110° C. Further optimization of digestion will be undertaken by digesting different samples each with the above fixed concentration of Proteinase K at 37° C. for varying time periods. The digestions will be stopped at 110° C. for 2 minutes. This will be followed by doing an in situ PCR with any house-keeping primers like GAPDH or β-actin. The digestion time that gives the strongest signal in maximum number of nuclei is the optimal digestion time.

After the standardization of enzyme digestions, in situ PCR will be carried out. In situ PCR will be followed by counterstaining the samples with hematoxylin for determination of sub-cellular localization of the amplified products. Samples will be imaged with a Zeiss LSM 510 confocal microscope equipped with Ar and He/Ne lasers. Samples will be excited at 633 nm and images collected with a 650 nm emission filter in the light path. All images will be collected using standardized laser intensities and photomultiplier tube settings for amplification and dark levels. All images will be processed with Adobe Photoshop.

1. A kit for screening biological samples for the presence telomere fusions, said kit comprising,
a PCR primer comprising the sequence of SEQ ID NO: 19; and
a reagent for conducting PCR reactions.

2. The kit of claim 1 wherein the PCR primer further comprises a restriction endonuclease recognition sequence covalently linked to the 5′ end of the sequence of SEQ IDNO: 19.

3. The kit of claim 1 wherein the PCR primer comprises a sequence represented by the general formula X-Y-(Z)ₙ, wherein
X represents the sequence of (SEQ ID NO: 44);
Y represents a restriction endonuclease recognition sequence;
Z represents the sequence of SEQ ID NO: 19; and
n is an integer selected from the range of 1-6.

4. The kit of claim 3 wherein n is 2 or 3

5. The kit of claim 1 wherein the PCR primer consists of SEQ ID NO: 21.

6. The kit of claim 1 wherein the reagents comprise a thermostable polymerase.

7. A purified nucleic acid sequence comprising SEQ ID NO: 19 and a restriction endonuclease recognition sequence, wherein the restriction endonuclease recognition sequence is covalently linked to the 5′ end of SEQ ID NO: 19.

8. The nucleic acid sequence of claim 7, wherein said sequence comprises the sequence of SEQ ID NO: 20.

9. A method of detecting telomere fusions in a biological sample, said method comprising
contacting cellular DNA isolated from said biological sample with a telomere specific PCR primer to form a reaction substrate;
conducting a PCR amplification reaction on the reaction substrate; and
detecting the presence of amplified products, wherein the detection of an amplified product indicates the presence of telomere fusions.

10. The method of claim 9 wherein the telomere specific PCR primer comprises the sequence of SEQ ID NO: 19.

11. The method of claim 10 wherein the telomere specific PCR primer comprises a restriction endonuclease recognition sequence covalently linked to the 5′ end of the sequence of SEQ ID NO: 19.

12. The method of claim 9 wherein the telomere specific PCR primer comprises a sequence represented by the general formula X-Y-(Z)ₙ, wherein
X represents the sequence of (SEQ ID NO: 44);
Y represents a restriction endonuclease recognition sequence;
Z represents the sequence of SEQ ID NO: 19; and
n is an integer selected from the range of 2-6.

13. The method of claim 9 wherein the telomere specific PCR primer consists of SEQ ID NO: 21.

14. The method of claim 9 wherein the biological sample comprises human breast tissue.

15. The method of claim 9 wherein the PCR amplification reaction is conducted on purified DNA from the cells of a patient.

16. The method of claim 9 wherein the PCR amplification reaction is conducted in situ on sectioned tissue obtained from a patient.

17. A method of detecting aberrant TRK2 expression in a tissue sample taken from a patient, said method comprising
contacting proteins of the patient's tissue with an ligand that specifically binds to TRK2;
detecting ligand-TRK2 complexes; and
comparing the expression of TRK2 protein to that of normal cells to detect aberrant TRK2 expression in the tissue sample.

18. The method of claim 17 wherein the ligand is an antibody.

19. The method of claim 18 wherein the step of contacting the proteins of the patient's tissue with an antibody comprises isolating total protein from the tissue sample, contacting the isolated protein with the antibody, and the detecting step comprises quantifying the amount of antibody specifically bound to the protein.

20. The method of claim 18 wherein the step of contacting the proteins of the patient's tissue with an antibody comprises preparing sections of the tissue sample, and incubating the tissue sections with the labeled antibody, and the detecting step comprises observing the cellular distribution of the specifically bound antibody.

21. A nucleic acid probe for detecting telomere fusions that are associated with neoplastic cells, wherein said probe comprises a nucleic acid sequence selected from the group consisting of SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57 and SEQ ID NO: 58, wherein said nucleic acid sequence is labeled with a reporter marker.

22. A method of detecting telomere fusions associated with neoplastic cells, said method comprising contacting nucleic acid sequences of a biological sample with a nucleic acid sequence selected from the group consisting of SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57 and SEQ ID NO: 58, wherein said nucleic acid sequence is labeled with a reporter marker.

23. The method of claim 22 wherein the nucleic acid sequences are purified from the biological sample prior to being contacted with the labeled nucleic acid sequence.

24. The method of claim 22 wherein the biological sample is cut into sections and the sectioned tissue is contacted with the labeled nucleic acid sequences.
